(19) United States
(12) Patent Application Publication
(10) Pub. No.: US 2016/0256141 A1
(43) Pub. Date: Sep. 8, 2016

(54) Multi-Vessel Closure System and Methods of Closing Vessels

(71) Applicant: TransCaval Solutions, Inc., Miami, FL (US)

(72) Inventors: Max Pierre Mendez, Miami, FL (US); Pedro Martinez-Clark, Miami, FL (US)

(73) Assignee: TransCaval Solutions, Inc., Miami, FL (US)

(21) Appl. No.: 15/060,960

(22) Filed: Mar. 4, 2016

Related U.S. Application Data
(60) Provisional application No. 62/128,320, filed on Mar. 4, 2015.

(51) Int. Cl. A61B 17/00; A61B 18/14

(52) U.S. Cl. CPC A61B 17/0057; A61B 18/1492; A61B 2018/00595

(57) ABSTRACT

A vessel occluding assembly includes first and second joined vessel aperture occluders each having a vessel aperture outer contact surface that, when one of the occluders is installed in a vessel aperture, hemostasis of a respective vessels is achieved, and a flexible tether connecting at the first and second occluders together such that, when the two occluders are implanted in a respectively vessel orifice, the occluders and the tether achieve sequential hemostasis of the plurality of vessels independent of relative tensions between the vessels.

### CROSS-REFERENCE TO RELATED APPLICATION

This application claims benefit of U.S. Provisional App. No. 62/128,320, filed Mar. 4, 2015, which is hereby incorporated by reference herein in its entirety.

### BACKGROUND OF THE INVENTION

1. Field of the Invention

The present invention lies in the field of vascular and tissue closure devices. This invention relates generally to occlusion devices and methods for the closure of multi-vessel apertures, caused by venous-arterial access. The invention also relates to delivery systems and mechanisms for such devices as well as devices that reduce procedural complexities and risks.

2. State of the Art

Complete percutaneous access into the atrial system up to the heart is desired. Limiting factors to this are arteries that do not facilitate current devices because of vessels that are atherosclerotic, tortuous, have a small diameter, are calcinated, or have percaline internal vascular walls. Anatomically parallel to the atrial system is the venous system, which does not typically have the same limiting properties. Percutaneous access into the venous system into the atrial system is advantageous and has been demonstrated and most impactful in caval-aortic procedures.

Transcaval access is a new catheter technique that enables non-surgical introduction of large devices, such as transcatheter heart valves, into the abdominal aorta. The resulting caval-aortic fistula is closed with a commercial nitinol occluder device that is an off-label use. Such occluders have important limitations, such as residual bleeding and theorized potential complications. Transcaval access (TCA) has been performed successfully in dozens of patients to date.

The transfemoral (TF) arterial approach is the most commonly utilized approach for transcatheter aortic valve replacement (TAVR). However, approximately 30% of screened patients are not suited for the TF approach because of peripheral arterial disease and a small caliber of their femoral arteries. The available alternatives are transapical for the Edwards Sapien valve (Edwards Lifesciences, Irvine, Calif.), subclavian/axillary for the self-expandable Medtronic CoreValve ReValving system (CV) (Medtronic, Minneapolis, Minn.), and transaortic for both prostheses. When compared to the TF approach, these alternative access options have a steep learning curve and are associated with significantly higher mortality and morbidity. The TF approach, on the other hand, is also associated with a significantly higher rate of vascular complications (up to 16%) when compared with other approaches. In addition, more than 3% of patients with symptomatic severe aortic stenosis are believed to have anatomic or physiological features making none of these approaches feasible.

It is because of these limitations in the existing approaches and technology that the transcaval approach was developed. The main drawback of the transcaval approach is access, making the patients susceptible to major bleeding complications. There are no available purpose-specific devices for closure of the caval-aorto tract that is created during the procedure. Operators have made off-label use of nitinol occluder devices marketed to close ductus arteriosus (Amplatzer Duct Occluder, St. Jude Medical, St. Paul, Minn.) or intracardiac defects (Amplatzer muscular VSD occluder) using the accompanying delivery system inside the TAVR sheath. Experience reveals several drawbacks associated with this off-label use of occluders and up to 79% of patients undergoing TAVR via transcaval approach have required blood transfusions. Once the issues with access closure (the only limitation) are resolved by development of a purpose specific caval-aortic occluder, this approach can serve as an alternative for all non-transfemoral approaches that currently constitute nearly half of the TAVR market. In fact, with the availability of an effective, reproducible, and predictable aorto-caval occluder, the trans-caval approach could be studied in a clinical trial against traditional trans-femoral arterial access. There are a number of patients that have a high anatomical bifurcation in the common femoral artery to the superficial and profunda femoral artery. This anatomical situation exposes the patient to an increased risk of vascular complications due to placement of a large arterial sheath at the bifurcation or at the proximal third of the superficial femoral artery. Even without a high femoral artery bifurcation, the common femoral artery measures less than 8 mm in most elderly individuals. Access in the distal aorta, as it is the case with the TCA, offers a much larger arterial surface with less vessel trauma when compared to the common femoral artery. The only true limitation of the TCA is an ability to successfully close the aorto-caval communication with total and immediate hemostasis.

In summary, off-label use devices lack immediate hemostasis. This results in a need for blood transfusions. Hemostasis assessment can only be conducted with a detached device and no bailout mechanism (i.e., attached retrieval mechanism) and can result in a need for re-intervention or blood transfusions. Off-label use devices impose severely unnatural stresses and strains onto vascular anatomy that is known to cause chronic damage and may result in full dissection and ambulatory hemorrhaging. Off-label use devises do not include safety mechanism that can prevent procedural accidental hemorrhaging.

### SUMMARY OF THE INVENTION

The invention provides systems and methods of vessel occlusion that overcome the previously-mentioned disadvantages of the heretofore-known devices and methods of this general type and that accomplish independent and sequential vascular hemostasis in a plurality of vessels by specifically designed occluders that do not rely on the relative tensions between vessels to create hemostasis. The invention also provides the ability to immediately assess hemostasis as well as provide features to increase safety and reduce risk of multi-vessel closure procedures.

One exemplary system and method herein utilizes a set of occluders (“an occluder set”) that contains two occluders connected by a tether. As used herein, an “occluder” is a device that is configured to close a vascular aperture. An occluder is defined by a generally circular structure that is equal to or larger than an aperture area and is composed of a structural frame and a sealing material extending at least about a circumference of the frame. Its structure can be determined by a shape memory alloy lattice that is shape set to a predetermined shape that interferes with targeted vessel geometries in order to maintain opposition of sealing surfaces and is held in place by inherent forces independently of a neighboring occluder. The occluder set can be asymmetric in shape to allow each occluder to conform to specific vascular properties. A tether is defined by a physical member between the occluders. Both occluders and tether have a normally expanded state, a partially expanded, and a collapsed state. The occluder set is in its collapsed state for delivery to the implantation site and/or to fit or pass through an aperture during implantation. The occluder set is in its partially expanded state during implantation, and is in its expanded state after implantation is complete. The occluders achieve successful hemostasis independently and do not rely on tension between the occluders, particularly at the tether, to maintain hemostasis. Thus, the tether can be slack when hemostasis is achieved. Herein, a partially expanded state is defined as a transition between a collapsed state and a fully expanded state. In the expanded state, the structure is larger in diameter than the vessel aperture and, therefore, prevents unintentional pull through after passage through an aperture and allows for visual and tactile indication of internal vessel wall contact. An occluder frame material can be metallic alloys or other known rigid, elastic and biocompatible materials.

The prior art devices have failed because they have been designed for a single cardiac tissue wall aperture occlusion and were not designed for multiple vessel occlusion with natural dimensions and geometries that are very different than single cardiac tissue walls. As a result, vessels having such implants suffer stresses and strains that are far beyond natural conditions and are known to cause complications and require further intervention. Multi-vessel occlusion procedures are new and the severity of long-term unnatural conditions is not fully understood. Additionally the sealing materials used in prior art allows for immediate blood pass through and eventual clotting and endothelial growth to complete hemostasis at an inadequate duration. In contrast, the configurations described herein include occlusion platforms that are purpose designed and impose minimal unnatural stresses and strains as well as facilitate immediate hemostasis by the use of impermeable materials.

In greater detail, the tether is a member that connects occluders together. The tether can be made as a fixed extension of the occluder frame. It can also be a different material as compared to the frame. Examples of materials include, but are not limited to, shape memory alloys, stainless steel, bio-absorbable materials, polymeric materials, fiber materials, polyester, polyurethane, PTFT, ePTFE, and other known bio-compatible materials. To have the tether translate from different states and dimensional conditions, it can be shaped as a coil, a cable, a loose cord, a corrugated tube, telescoping tubes, or a compliant beam shape. The tether can be selectably attachable or fixed by crimping, press-fitting, bonding, threading, or various welded attachment methods. Alternatively, the tether member can be made of a tubular impermeable material and have an open connection at each occluder to create a hemostatic connection between both vessels.

It is standard practice for a guidewire to be placed through the vessel aperture path to maintain a physical track that facilitates continuing pass through up until full determination of successful procedure. Sealing modalities used in prior art devices are not designed for parallel guidewires or additional physical members and, as a result, immediate hemostasis evaluation becomes impossible. Significantly, full hemostasis evaluation cannot be gained until the parallel guidewire is removed, at which point there is no physical track to re-enter the vessel aperture. This situation poses a high risk, which is avoided by the systems and methods described herein by providing a sealing modality that is independent of the procedural guidewire. In one exemplary embodiment, the occluders contain an inboard guidewire lumen that maintains the guidewire from impeding sealing surfaces and allows for accurate and immediate hemostasis assessment even before the guidewire is removed. The lumen is configured to automatically close by a preloaded cover, by clotting or by endothelial growth. The delivery tube assembly can be a multi-shaped lumen to provide paths for both a delivery cable and a guidewire. It can also extend into the occluder area to allow for keying of the occluder during loading to automatically align guidewire paths of the delivery system and the occluder. Alternatively, a catheter introducer sheath used during Transcatheter Aortic Valve Replacement (TAVR) implantation can deliver on-board occluders before or after TAVR implantation. Occluders can be loaded onto existing introducers sheaths or on a proprietary purpose built sheath device.

It would be advantageous to use the same occlusion platform as multi-vessel closure procedures progress and as new locations are discovered. The occlusion devices and methods described herein require minor changes to comply with different aperture locations and are, therefore, independent of future research in the field of multi-vessel closure.

In any preset structure embodiment, a structure frame can be form-fitting to not apply stresses to vessel aperture surfaces. An intentionally undersized and non-interfering frame design has a sealing member that is force-fitting and is able to conform to vascular surfaces. Soft spring-loaded materials in an uninterrupted member, such as a disk of foam, are able to completely conform to irregular surfaces because of their continuous number of contact points. A combination of sparse spring loaded frame points and a continuous compliant material increases cooptation with grossly irregular surfaces having a large topological height difference. A frame structure that houses a sealing member can be preloaded with additional sealing members or replaced with the best performing sealing member as determined by the operator. The sealing member can reside internally or externally to the occluder.

Another exemplary system utilizes occluders with vessel matching geometries that allow vessels to more closely resemble their natural geometries after implantation, thereby; reducing complications attributed to unnatural vessel manipulation. Vessel aperture geometry is not radially uniform about its central axis because its central axis is perpendicular to the vessel central axis and, as a result, the circular diameter is overlayed on an arced tubular vessel surface, thereby altering the vessel aperture with respect to the opening tool. Similarly to the described vessel aperture geometry, an occluder frame structure can have an arced radial profile that is perpendicular to its central axis. A vessel matching occluder is not rotationally uniform and radiopaque markers can be positioned to indicate correct rotational relationship to the operator. The delivery system and the occluder can contain rotational keying and aligning features to maintain correct relative relationships with alignment markers. A loading device can be used during loading to aid operators. Additionally, features located on the internal side can interface with blood flow and control automatic rotational alignment.

In greater detail, the connection member serves as a temporary attachment between the operator and the implant. The connection member can be a mechanical interlock joint that is disengaged when specific forces are transmitted from the operator handle to the connection member. In one example, the joint is a press-fit joint. The connection member can also be a threaded joint that is disengaged only when a specific torque is transmitted from the operator handle to the threaded connection member. The connection function can be engaged and disengaged by a set of members that complete connection in engaged state and allow disengagement when they are translated with respect to each other. The connection can be one fixed joint that relies on forces that exceed extreme procedural forces in order to fracture a stress concentration area. The connection can also be biodegradable and dissolve and separate at an acceptable timeframe. Additionally, the connection member can be designed to articulate by using a universal joint mechanism or a spring support mechanism that allows for a free range of angular rotation in order to passively comply with varying deployment tube and aperture axis angles. The connection can also be made by using a locking pin and release operation. A flexible cord can be used as a pin, and thereafter cut and removed at the device handle. This method poses no need for rotation or torque.

In greater detail, the sealing member is compliant and is able to conform to vessel surfaces regardless of irregularities. It can be external of the frame structure and contact vessel/tissue walls to create hemostasis by filling volume in between the disk frame and the vascular/tissue wall. The sealing member can be made from DACRON®, PET, PTFE, ePTFE, an epoxy bladder, foam, a mesh, composites of different materials, and other known biocompatible materials. Sealing performance can rely on compression from the occluder structure or can be independent. The sealing surface can have a raised area, such as a perimeter bead, to increase compression at those specific areas. Depending on the procedure being performed, the occluder can be covered by different polymers or by a matrix or mesh of material. The covering can be semi-porous for sealing over time with cellular in-growth and/or it can have portions that are non-porous to seal immediately upon implantation or even just before implantation. A non-porous covering over the entirety is also contemplated. For example, an occlusion curtain can be disposed within the cross-section of the central orifice, in particular, within the waist, dependent on the effect that is desired. It can be beneficial if the material used is distensible so that it does not corrugate or pleat but, in particular circumstances, it can be non-distensible.

In detail, the delivery system can be composed of a delivery tube and a delivery member and maintain the occluder set in a collapsed state by delivery member attachment and delivery tube encapsulation. An expanded occluder set can be actuated to a collapsed state by an operator pulling the delivery member through delivery tube, thereby pulling the occluder set into the delivery tube. Translation of the occluder can also be driven by a mix of directional dynamic mechanisms, such as a handle rotation member to translate linear motion at a distal end of the delivery tube. The occluder set also can be driven to its collapsed state by a tube that is pushed over its exterior surface. Alternatively, the delivery system can have mechanisms, such as linkages, purse strings, or control members, to actuate occluders into expanded or collapsed configurations. The delivery tube and delivery system components along the length of the system can have variable diameters to reduce contact and stress to vasculature tract. For example, the distal diameter of the delivery tube can similar to a collapsed occluder assembly diameter where the proximal section can have a similar diameter to the diameter of the delivery member. The delivery system may also contain a channel for a guidewire support tube. The handle of the delivery system can contain features to facilitate system flushing, seals to prevent blood exiting the device, locks and valves to seal components that translate within or out of the handle. Operator controlled components can have grip sections, geometries and mechanical advantage sections to reduce fatigue during device operation.

The deployment tube distal end can be angled or curved so that its central axis closely approaches the central axis of the vessel aperture in order to reduce the level of unnatural forces onto vessels and to improve the accuracy of occluder hemostasis and assessment before implantation. During implantation, the occluder is maintained in a partial expansion state within the vessel is then tensioned until tactile and visual feedback of the occluder to vessel contact is observed. Alternatively, the deployment tube can be terminated at an angle with respect to a circular cross-section and that is closely aligned with the central axis of the vessel. The resultant deployment tube opening profile will closely match vessel aperture and allow for an uneven deployment of occluder that will better match natural misalignment. These features can be manipulated into optimal orientations with the assistance of visual alignment markers. Alternatively, the distal end of the deployment tube can actively or passively articulate to better align the occluder exit axis with the aperture axis.

An asymmetric set of uneven occluders can be used to closely match vessel specific geometries to increase hemostasis and reduce damage to native vessels. It is known that abdominal aortas have a relatively smaller diameter and thicker walls and are more prone to disease than an inferior vena cava. Thus, an occluder catered to each vessel is advantageous. Occluder designation can be indicated to operators by colored labels, such as a suture or thread used to join the sealing member to the frame. Blue is typically reserved for the venous system and red is typically used for the arterial system and these are options for use with the present systems and methods.

Several zero-waist section occluder structures are identified to yield an occluder that is independent of wall thicknesses and conforms to a large range of wall thicknesses starting from zero. Prior art devices are specifically designed to be implanted within cardiac tissue walls that are known to be thicker and tougher than vessels. Prior art devices are formed from shape memory nitinol braided structures and have a specific waist section as defined by a portion of the occluder that resides within an aperture that are longer than vascular thicknesses. Zero waist length is advantageous for thin vessels but is difficult to shape set using braided tubular structures because typical shape memory forming processes use mandrels that dictate profile. A lost wire wrap method is used to create a zero-length waist section. Shape setting is performed in at least two steps where a fine wire is used to constraint the braid in a tight waist section. Once the waist section is created, the wire is removed and the remaining structure is compressed to close the waist gap during heat setting. In some cases, this method will introduce inadequate strain levels to the shape set material. Alternatively, a woven nitinol lattice can be created to yield a zero waist section by alternating strands crossing a central perpendicular axis in a diagonal fashion. This configuration will yield some strands that are located in both sides of the waist central section. Alternatively, mechanical joint methods with pivots do not suffer bending strains and can be designed to create zero-waist lengths.

Inadvertent pull through of a catheter is not well tolerated in many procedures. The issue is exaggerated during a vessel closure operation because of hemorrhaging. Inadvertent pull through is associated with accidents. The systems and methods can have an anti-pull out mechanism that reduces the risk of accidental hemorrhaging. A system can be implemented that allows for two-handed manipulation of the device. Catheter based operators usually ground one hand to the access sheath and the other hand on the catheter device. Instead of the operator grounding on the access sheath, the user can disengage a system that normally locks the catheter, thereby adding another level of involvement towards an accidental pull through. This lock disengagement can be located on the delivery system structure or the grounding structure. The system also can be self-driven to detect when an accident condition has happened and apply a lock in that circumstance. For example, a sensor similar to a computer mouse can detect catheter movement and, when movement exceeds a preset rate, the system can engage a lock. Alternatively, this smart lock can be engaged by other kinds of user commands such as voice. The lock can be spring loaded, balloon inflated, or driven. Similarly, this mechanism can be implemented into an onboard configuration and interface with introducer sheath to provide relative locking.

Although the invention is illustrated and described herein as embodied in systems and methods of multi-vessel closure, it is, nevertheless, not intended to be limited to the details shown because various modifications and structural changes may be made therein without departing from the spirit of the invention and within the scope and range of equivalents of the claims. By way of example, the structure of the individual occluders, alone or in combination with the deployment systems taught herein, can be used to seal and provide hemostasis at an aperture in a single tissue wall, including in a vessel, or in the wall of an organ, such as the heart, and more particularly, by way of example only, to treat atrial septal defects. Additionally, well-known elements of exemplary embodiments of the invention will not be described in detail or will be omitted so as not to obscure the relevant details of the invention.

Additional advantages and other features characteristic of the present invention will be set forth in the detailed description that follows and may be apparent from the detailed description or may be learned by practice of exemplary embodiments of the invention. Still other advantages of the invention may be realized by any of the instrumentalities, methods, or combinations particularly pointed out in the claims.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing Figures, in which like reference numerals are carried forward.

### BRIEF DESCRIPTION OF THE DRAWINGS

The accompanying figures, where like reference numerals refer to identical or functionally similar elements throughout the separate views, which are not true to scale, and which, together with the detailed description below, are incorporated in and form part of the specification, serve to illustrate further various embodiments and to explain various principles and advantages all in accordance with the present invention. Advantages of embodiments of the present invention will be apparent from the following detailed description of the exemplary embodiments thereof, which description should be considered in conjunction with the accompanying drawings in which:

FIG. 1 is a fragmentary illustration of a human vascular system;

FIG. 2 is a fragmentary illustration of a human vascular system including a superimposed conduit demonstrating a transcaval path from the femoral vein up to heart;

FIG. 3 is an anterior fluoroscopic image of a transcaval access procedural step;

FIG. 4 is a diagrammatic representation of FIG. 3;

FIG. 5 is an anterior fluoroscopic image similar to FIG. 3 of a transcaval access procedural step;

FIG. 6 is a diagrammatic representation of FIG. 5;

FIG. 7 is a fragmentary, cross-sectional normal view of a vessel aperture;

FIG. 8 is a fragmentary, diagrammatic representation of an anterior view of vessels with apertures including a representation of blood flow path;

FIG. 9 is a fragmentary, perspective view of FIG. 8.

FIG. 10 is a fragmentary, side cross-sectional, diagrammatic view of an implanted occluder set within two vessel apertures;

FIG. 11 is a fragmentary, perspective view of FIG. 10;

FIG. 12 is a fragmentary, side cross-sectional, diagrammatic view of an implanted occluder set attached to delivery system within two vessel apertures;

FIG. 13 is a fragmentary, side cross-sectional, diagrammatic view of a collapsed occluder set within two vessel apertures;

FIG. 14 is a fragmentary, partially cross-sectional, perspective view of FIG. 13;

FIG. 15 is a fragmentary, partially cross-sectional, perspective view of FIG. 12;

FIGS. 16A-16F are fragmentary, side cross-sectional, diagrammatic views of the sequential implantation process of a non-tensioning occluder set into two vessel apertures;

FIGS. 17A-17D are fragmentary, side cross-sectional, diagrammatic views of the sequential implantation process of a tensioning prior-art occluder set into two vessel apertures;

FIG. 18 is a fragmentary, side cross-sectional, diagrammatic view of a nominal prior art occluder superimposed into the interstitial space between a scaled representation of vessel apertures;

FIG. 19 is a fragmentary, side cross-sectional, diagrammatic view of an implanted prior art occluder into two vessel apertures;

FIG. 20 is a fragmentary, cross-sectional, perspective view of FIG. 19;

FIG. 21 is a fragmentary, side cross-sectional, diagrammatic view of an implanted prior art occluder into a single vessel aperture;

FIG. 22 is a fragmentary, cross-sectional, perspective view of FIG. 21;

FIG. 23 is a fragmentary, perspective view of a woven mesh structure;

FIG. 24 is a fragmentary, perspective view of a sectioned tubular woven mesh structure;

FIG. 25 is a fragmentary, perspective view of tubular machined structure;

FIG. 26 is a fragmentary, side partial cross-sectional, diagrammatic view of an implanted occluder set attached to a curved delivery system within two vessel apertures;

FIG. 27 is a fragmentary, side partial cross-sectional, diagrammatic view of a partially implanted occluder set attached to a delivery system with an angled opening within two vessel apertures;

FIG. 28 is a fragmentary, side cross-sectional, diagrammatic view of an implanted occluder set including sealing skirts within two vessel apertures;

FIG. 29 is a fragmentary, diagrammatic side view a single implanted occluder into a single vessel wall with surface irregularities;

FIG. 30 is a fragmentary, diagrammatic frontal, angled view of a sealing skirt overlayed onto surface irregularities;

FIG. 31 is similar to FIG. 30 and shows sealing skirt separated for clarity;

FIG. 32 is a fragmentary, cross-sectional, diagrammatic, side view of a single implanted occluder with a reentry port within a vessel aperture;

FIG. 33 is a fragmentary, cross-sectional, diagrammatic, side view of a single implanted occluder with a reentry port and reentry plug removal feature within a vessel aperture;

FIG. 34 is a fragmentary, cross-sectional, diagrammatic, side view of a single implanted occluder with a reentry port and removed reentry plug within a vessel aperture;

FIG. 35 is a fragmentary, frontal view of an implanted prior art occluder with a parallel guidewire;

FIG. 36 is a fragmentary, cross-sectional, perspective view of FIG. 35;

FIG. 37 is a fragmentary, side cross-sectional, diagrammatic view of an implanted occluder set including a central guidewire within two vessel apertures;

FIG. 38 is a fragmentary, side cross-sectional, diagrammatic view of an collapsed occluder set including a central guidewire;

FIG. 39 is a frontal view of an expanded occluder with a central guidewire lumen;

FIG. 40 is a fragmentary, cross-sectional, perspective view of FIG. 38;

FIG. 41 is a fragmentary, cross-sectional, perspective view of FIG. 37;

FIG. 42 is a frontal view of an expanded occluder with an open central guidewire channel;

FIG. 43 is a frontal view of an expanded occluder with a closed central guidewire channel;

FIG. 44 is an anterior CT image of severely diseased aortic vessels;

FIG. 45 is a frontal view of a flat frame occluder;

FIG. 46 is a side view of FIG. 45.

FIG. 47 is a perspective and semi-transparent view of a single implanted flat frame occluder within a vessel aperture;

FIG. 48 is a fragmentary, cross-sectional, side view of a collapsed flat frame occluder set;

FIG. 49 is a fragmentary, cross-sectional, side view of a single collapsed flat frame occluder 304 within a vessel wall aperture;

FIG. 50 is a fragmentary, cross-sectional, side view of a single partially expanded flat frame occluder within a vessel wall aperture;

FIG. 51 is a fragmentary, cross-sectional, side view of a single partially expanded flat frame occluder within a vessel wall aperture;

FIG. 52 is a fragmentary, cross-sectional, side view of a single expanded flat frame occluder within a vessel wall aperture;

FIG. 53 is a semi-transparent, perspective view of a partially expanded flat frame occluder with sealing members;

FIG. 54 is a cross-sectional view of FIG. 53;

FIG. 55A is a side view of FIG. 53, shown within a vessel wall aperture;

FIG. 55B is a side view of FIG. 54, shown within a vessel wall aperture;

FIG. 56 is a cross-sectional view of an expanded flat frame occluder with sealing members, shown in a nominally flat position;

FIG. 57 is a fragmentary, perspective view a collapsed flat beam occluder with sealing members;

FIG. 58 is a fragmentary, cross-sectional view of FIG. 57;

FIG. 59 is a side view of a collapsed tubular beam occluder;

FIG. 60 is a side view of a partially expanded tubular beam occluder;

FIG. 61 is a side view of a fully expanded tubular beam occluder;

FIG. 62 is a cross-sectional, side view of a partially expanded tubular beam occluder with sealing members;

FIG. 63 is a perspective view of FIG. 60;

FIG. 64 is a perspective view of FIG. 61;

FIG. 65 is a frontal view of FIG. 61;

FIG. 66 is a cross-sectional side view of a single occluder with zero waist length;

FIG. 67 is a fragmentary, cross-sectional side view of a single occluder with zero waist length superimposed over a single vessel aperture;

FIG. 68 is a fragmentary, cross-sectional side view of a single implanted occluder with zero waist length into a single vessel aperture;

FIG. 69 is a fragmentary, cross-sectional side view of an implanted occluder set with a sensor;

FIG. 70 is fragmentary, perspective view of FIG. 69;

FIG. 71 is a fragmentary, cross-sectional, side view of a single collapsed occluder with a sheath reentry port, within an introducer sheath and inserted into a vessel wall aperture;

FIG. 72 is a fragmentary, cross-sectional, side view of a single partially expanded occluder with a sheath reentry port;

FIG. 73 is a fragmentary, cross-sectional, side view of a single implanted occluder with a sheath reentry port;

FIG. 74 is a fragmentary, cross-sectional, side view of a single implanted occluder with a reentry plug removed and introducer sheath passing through the occluder central port;

FIG. 75 is a fragmentary, cross-sectional, side view of a collapsed introducer sheath loaded occluder creating an aperture;

FIG. 76 is a fragmentary, cross-sectional, side view of the occluder from FIG. 75 and in a correct implantation location that is central to vessel wall;

FIG. 77 is a fragmentary, cross-sectional, side view of an expanded introducer sheath loaded occluder;

FIG. 78 is a fragmentary, partial cross-sectional, angled side view of FIG. 77;

FIG. 79 is a fragmentary, cross-sectional, side view of an expanded introducer sheath loaded occluder with an introducer sheath through central occluder port;

FIG. 80 is a perspective view of a non-circular occluder set;

FIG. 81 is a fragmentary, partially cross-sectioned, perspective view of an implanted non-circular occluder set within vessel apertures;

FIG. 82 is a fragmentary, perspective view of an implanted non-circular occluder set within vessel apertures;

FIG. 83 is a top view of FIG. 80;

FIG. 84 is a cross-sectional view of FIG. 83;

FIG. 85 is a side view of FIG. 80;

FIG. 86 is a fragmentary illustration of a human vascular system including a superimposed conduit demonstrating a transcaval path from out of body, through skin, into femoral vein, through transcaval access and into aorta;

FIG. 87 is a fragmentary, diagrammatic side view of an anti-pull out system;

FIG. 88 is a fragmentary, diagrammatic side view of an anti-pull out system;

FIG. 89 is a fragmentary illustration of a human vascular system including a superimposed guidewire following a transcaval path from femoral vein, through transcaval access and into aorta;

FIG. 90 is a fragmentary, top view of a performance guidewire;

FIG. 91 is a fragmentary, perspective view of an electrocautery guidewire adapter;

FIG. 92 is a fragmentary, partially cross-sectional, perspective view of a guide-catheter with support members within a vessel; and

FIG. 93 is a fragmentary, cross-sectional, side view of a guide-catheter with support members within a vessel.

FIG. 94 is a frontal view of a wire frame occluder;

FIG. 95 is a perspective view of a wire frame occluder;

FIG. 96 is a frontal, semi-transparent view of a wire frame occluder with sealing members;

FIG. 97 is a side view of a wire frame occluder with sealing members;

FIG. 98 is frontal view of a wire frame occluder with overlapping beam groups;

FIG. 99 is perspective view of a wire frame occluder with overlapping beam groups;

FIG. 100 is a perspective view of a wire frame occluder with continuous wire groups;

FIG. 101 is a perspective view of a wire frame occluder with continuous wire and overlapping groups;

FIG. 102 is a perspective view of a wire frame occluder with a continuous wire frame;

FIG. 103 is a perspective view of a wire frame occluder with continuous wire and overlapping groups;

FIG. 104 is a perspective view of a wire frame occluder with overlapping groups attached by a central retention member;

FIG. 105 is a perspective view of a wire frame occluder with overlapping groups;

FIG. 106 is a perspective view of a wire frame occluder with continuous wire and overlapping groups;

FIG. 107 is a perspective view of a wire frame occluder with continuous wire and overlapping groups;

FIG. 108A is a perspective view of an occluder set expanded into vessel aperture walls;

FIG. 108B is a fragmentary, side view of FIG. 108A and shows a proximal and distal occluder;

FIG. 108C is a fragmentary, side view of FIG. 108A and shows one side of a proximal occluder transitioning into a collapsed state;

FIG. 108D is a fragmentary, side view of FIG. 108C and shows one side of a proximal occluder collapse and another side transitioning into a collapsed state;

FIG. 108E is a fragmentary, side view of FIG. 108D and shows both sides of a proximal occluder in a collapsed state;

FIG. 108F is a fragmentary, side view of FIG. 108E and shows one side of a distal occluder transitioning into a collapsed state;

FIG. 108G is a fragmentary, side view of FIG. 108F and shows a distal occluder in a collapsed state;

FIG. 109 is a fragmentary, frontal view of an occluder expanded into a vessel wall.

FIG. 110 is a fragmentary, side view of FIG. 109.

FIG. 111 is a fragmentary, side view of a wire frame occluder.

FIG. 112 is a fragmentary, perspective view of a wire frame occluder;

FIG. 113 is a fragmentary, side view of a wire frame occluder with an attached delivery member;

FIG. 114 is a fragmentary, perspective view of a wire frame occluder with an articulated attachment member;

FIG. 115A is a fragmentary, frontal view of a wire frame occluder with a closed central guidewire lumen;

FIG. 115B is a fragmentary, frontal view of a wire frame occluder with an open central guidewire lumen;

FIG. 116 is a fragmentary, perspective view of a wire frame occluder with a guidewire support tube and guidewire within a central guidewire lumen;

FIG. 117 is a fragmentary, perspective view of a wire frame occluder set implanted into their respective vessels;

FIG. 118 is a fragmentary, perspective view of a wire frame occluder set implanted into vessel walls;

FIG. 119 is a fragmentary, cross-sectional, view of a collapsed occluder set within a delivery tube;

FIG. 120 is a fragmentary, perspective view of a collapsed occluder set.

### DETAILED DESCRIPTION OF THE PREFERRED EMBODIMENTS

As required, detailed embodiments of the systems and methods are disclosed herein; however, it is to be understood that the disclosed embodiments are merely exemplary of the systems and methods, which can be embodied in various forms. Therefore, specific structural and functional details disclosed herein are not to be interpreted as limiting, but merely as a basis for the claims and as a representative basis for teaching one skilled in the art to variously employ the systems and methods in virtually any appropriately detailed structure. Further, the terms and phrases used herein are not intended to be limiting; but rather, to provide an understandable description of the systems and methods. While the specification concludes with claims defining the features of the systems and methods that are regarded as novel, it is believed that the systems and methods will be better understood from a consideration of the following description in conjunction with the drawing figures, in which like reference numerals are carried forward.

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Alternate embodiments may be devised without departing from the spirit or the scope of the invention. Additionally, well-known elements of exemplary embodiments of the systems and methods will not be described in detail or will be omitted so as not to obscure the relevant details of the systems and methods.

Before the systems and methods are disclosed and described, it is to be understood that the terminology used herein is for the purpose of describing particular embodiments only and is not intended to be limiting. The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element. The terms “including” and/or “having,” as used herein, are defined as comprising (i.e., open language). The terms “a” or “an”, as used herein, are defined as one or more than one. The term “plurality,” as used herein, is defined as two or more than two. The term “another,” as used herein, is defined as at least a second or more. The description may use the terms “embodiment” or “embodiments,” which may each refer to one or more of the same or different embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact (e.g., directly coupled). However, “coupled” may also mean that two or more elements are not in direct contact with each other, but yet still cooperate or interact with each other (e.g., indirectly coupled).

For the purposes of the description, a phrase in the form “A/B” or in the form “A and/or B” or in the form “at least one of A and B” means (A), (B), or (A and B), where A and B are variables indicating a particular object or attribute. When used, this phrase is intended to and is hereby defined as a choice of A or B or both A and B, which is similar to the phrase “and/or”. Where more than two variables are present in such a phrase, this phrase is hereby defined as including only one of the variables, any one of the variables, any combination of any of the variables, and all of the variables, for example, a phrase in the form “at least one of A, B, and C” means (A), (B), (C), (A and B), (A and C), (B and C), or (A, B and C).

Relational terms such as first and second, top and bottom, and the like may be used solely to distinguish one entity or action from another entity or action without necessarily requiring or implying any actual such relationship or order between such entities or actions. The description may use perspective-based descriptions such as up/down, back/front, and top/bottom. Such descriptions are merely used to facilitate the discussion and are not intended to restrict the application of disclosed embodiments. Various operations may be described as multiple discrete operations in turn, in a manner that may be helpful in understanding embodiments; however, the order of description should not be construed to imply that these operations are order dependent.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure.

Herein various embodiments of the systems and methods are described. In many of the different embodiments, features are similar. Therefore, to avoid redundancy, repetitive description of these similar features may not be made in some circumstances. It shall be understood, however, that description of a first-appearing feature applies to the later described similar feature and each respective description, therefore, is to be incorporated therein without such repetition.

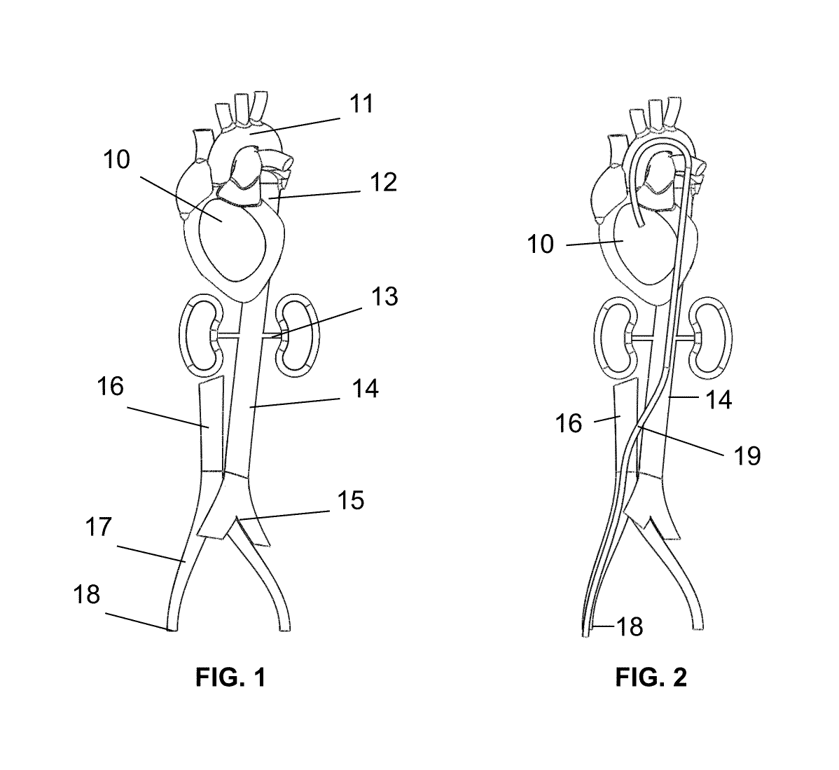

Described now are exemplary embodiments. Referring now to the figures of the drawings in detail and first, particularly to FIG. 1, there is an anterior view of the human aortic vascular system including the heart 10, the aortic arch 11, the thoracic aorta 12, the renal arteries 13, the abdominal aorta 14, and an aortic bifurcation 15. Also shown is a section of the venous vascular system including the inferior vena cava (IVC) 16, a venous bifurcation, the iliac vein 17, and the femoral vein 18.

FIG. 2 is a vascular diagram like that shown in FIG. 1 but with a superimposed conduit 19 demonstrating a path from the femoral vein 18, into the IVC, out of the IVC, into the abdominal aorta 14, and up to heart 10. This path exhibits a caval-aortic access.

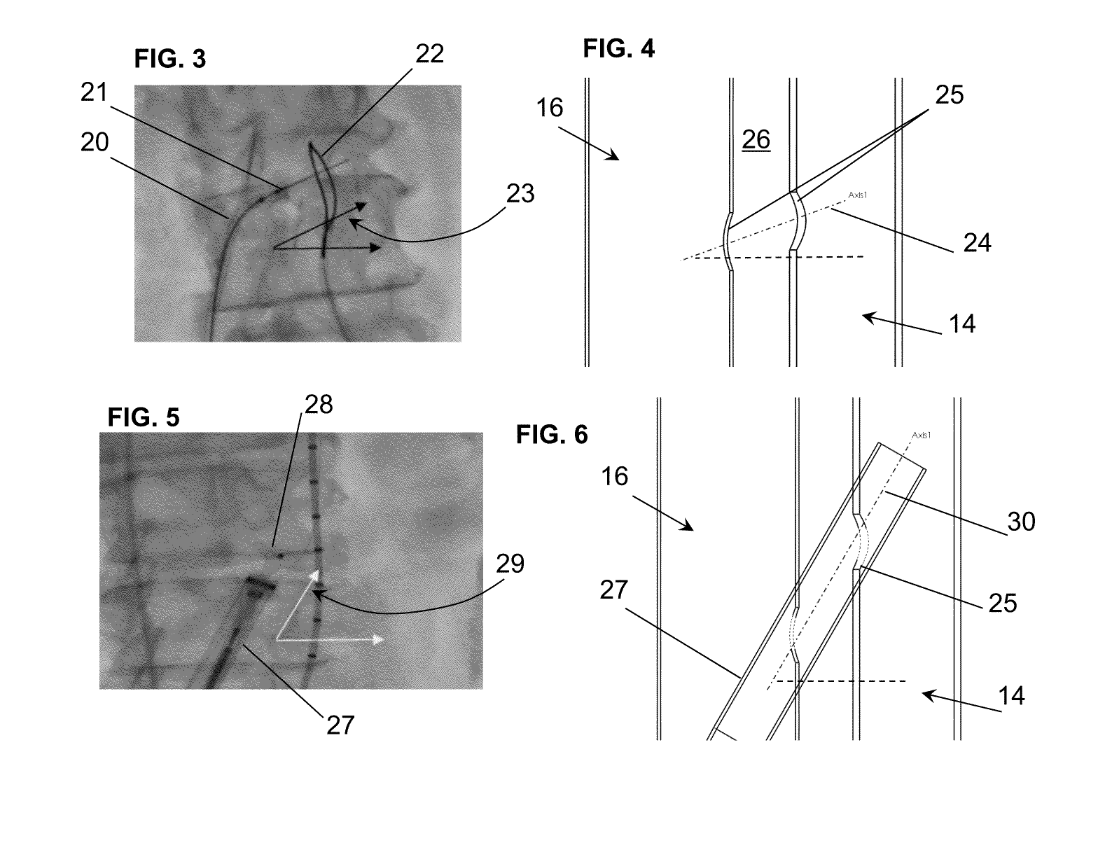

FIG. 3 is an anterior fluoroscopic image of a transcaval access into the abdominal aorta. It includes a guide catheter 20, a crossing guidewire 21, and a capture snare 22 that are used during crossing. The angle diagram 23 represents the initial transcaval crossing angle with respect to the transverse axis.

FIG. 4 is a diagrammatic representation of the anatomy of FIG. 3. It includes the IVC 16 and the abdominal aorta 14 as well as the initial transcaval crossing axis 24 and apertures 25 at diameters equal to the procedural introducer sheath. FIG. 4 also shows the interstitial space 26 between vessels.

FIG. 5 is an anterior fluoroscopic image similar to FIG. 3. It includes a procedural introducer sheath 27 with a presently off-label use occluder 28 in a semi-deployed position. The superimposed diagram 29 demonstrates the transcaval crossing angle when created by the procedural introducer sheath.

FIG. 6 is a diagrammatic representation of FIG. 5. FIG. 6 demonstrates the transcaval crossing axis 30 created by procedural introducer sheath.

The transcaval aperture angular range represented in FIGS. 5 and 6 demonstrates the need for a closure device that can conform to apertures created during the procedure and their locations as well as to allow for restoration of natural orientations.

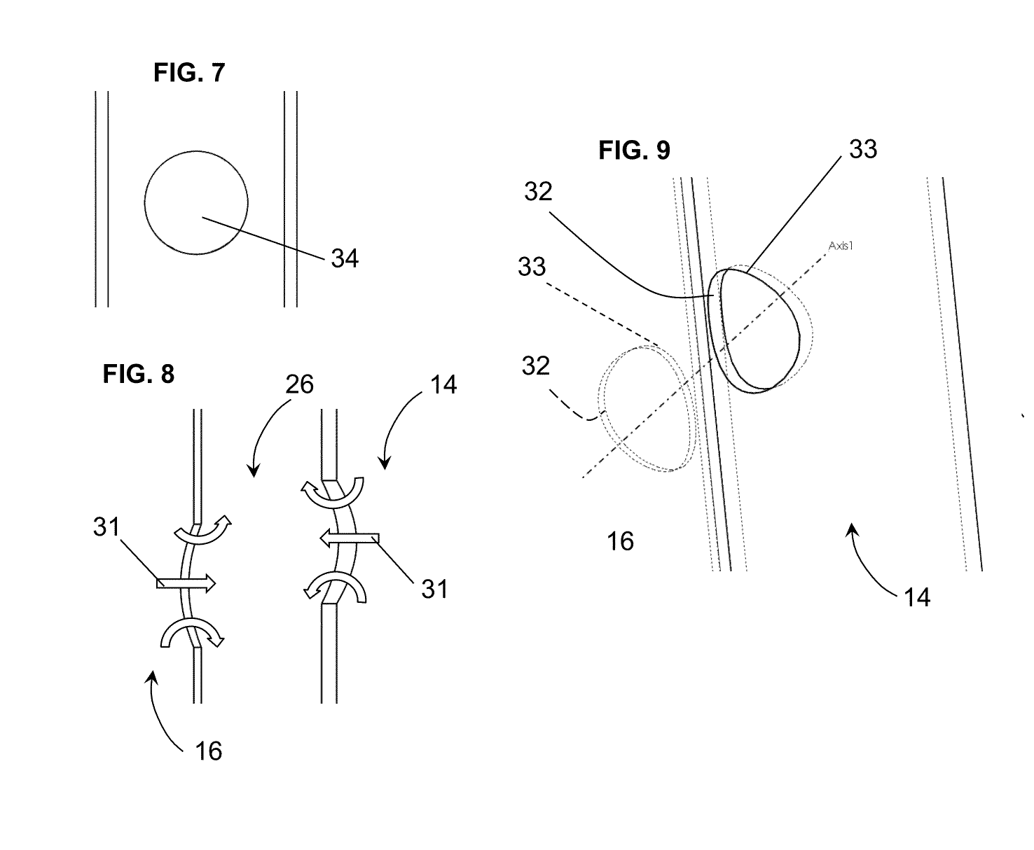

FIG. 7 is a fragmentary, cross-sectional normal view of an aperture and its internal area 34 created by transcaval access.

FIG. 8 is a fragmentary, diagrammatic representation of an anterior view of vessels with apertures including a representation of a blood flow path 31.

FIG. 9 is a fragmentary, perspective view of the diagrammatic vessel representation from FIG. 8. FIG. 9 includes the identified locations that need to be contacted to create hemostasis. These locations are the internal areas of the vessel wall aperture 32 as well as perimetral locations 33 internal and external of the vessels. These perimetral areas define the aperture area boundaries. Full hemostasis can be achieved by full occlusion of the aperture area up to its boundaries.

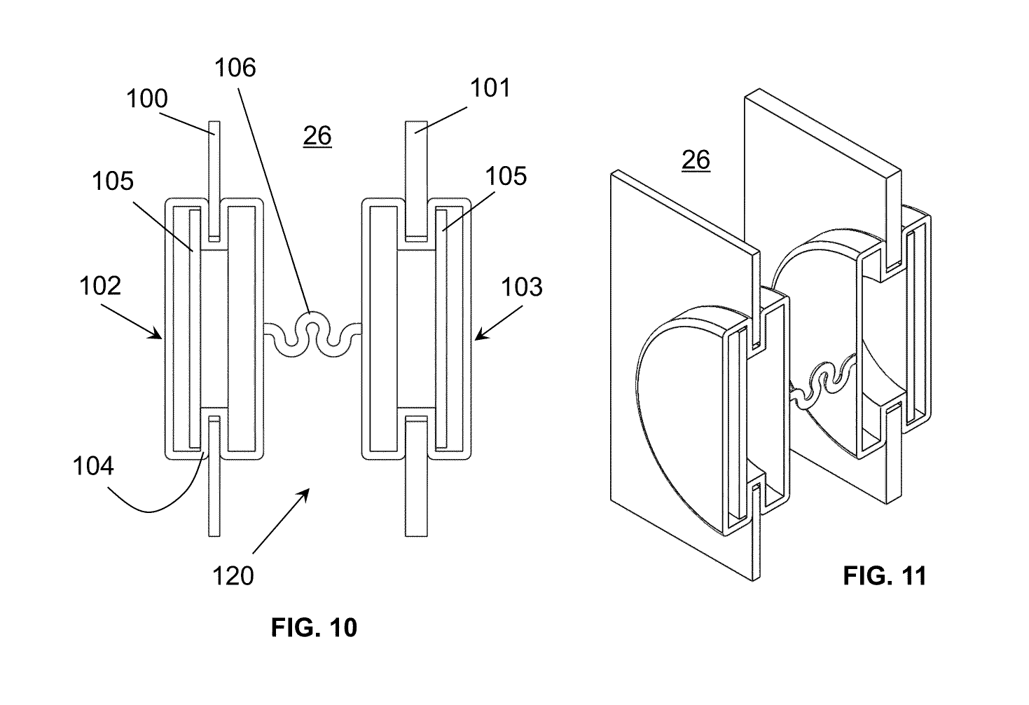

FIG. 10 is a diagrammatic representation of a cross-sectional side view of a linear and partial IVC vessel wall 100 and a linear and partial aortic vessel wall 101 with a respective IVC occluder 102 and an aortic occluder 103 in their implanted state. Each occluder 102, 103 has an expanded frame 104 that defines a structural perimeter catered to specific vessel wall aperture geometries as well as an expanded sealing member 105. The frame 104 and sealing member 105 work in unison to completely occlude the respective aperture areas up to its boundary. The occluders 102, 103 are physically connected by a tether member 106 (shown in an implanted state) that resides in the interstitial space 26. The composition of the occluders 102, 130 and the tether 106 define an expanded and implanted occluder set 120. FIG. 11 is a fragmentary, cross-sectional perspective view of FIG. 10.

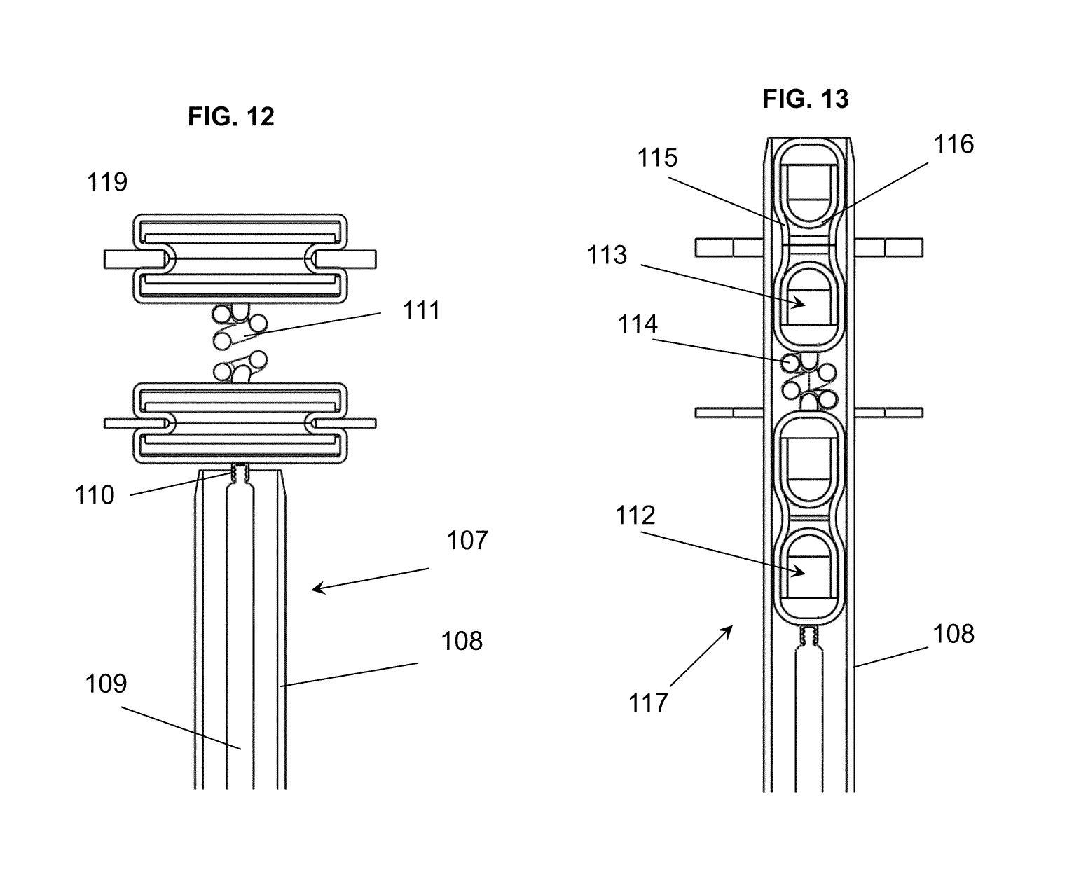

FIG. 12 is a fragmentary, side cross-sectional, diagrammatic view similar to FIG. 10 and shows an expanded occluder set in its expanded configuration but still attached to its delivery system. This orientation is defined as a fully expanded and attached occluder set 119 that is composed of a delivery tube 108 and a delivery member 109 that is selectably attached to occluder by a connection member 110. Selectable is defined herein as being selected by the user to be attached or to be unattached (i.e., removed). FIG. 12 also demonstrates a helical tether 111, similar to a helical spring, in its implanted position.

FIG. 13 is a fragmentary, side cross-sectional, diagrammatic view similar to FIG. 12 but with the occluder set 119 in a collapsed configuration 117. The occluders 112, 113 are shown in their collapsed state, which is in contrast with the occluders 102, 103 that are in an expanded state in FIG. 12. In greater detail, the collapsed occluders are composed of collapsed occluder frame 115, a collapsed sealing member 116, and a collapsed tether 114 and are constrained by the delivery tube 108.

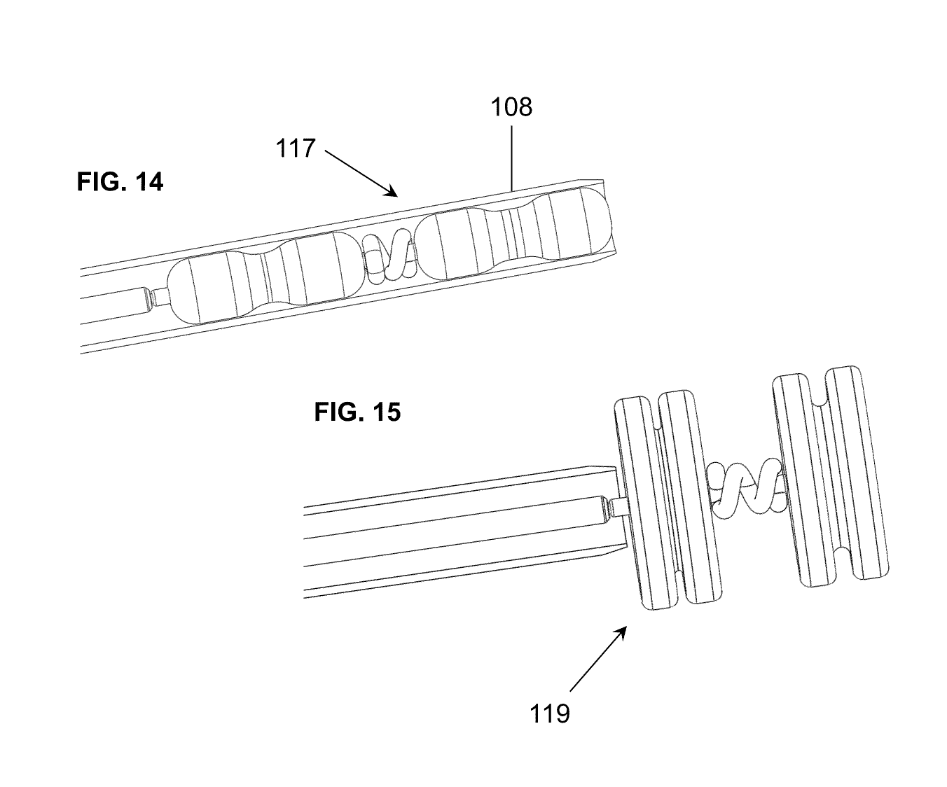

FIG. 14 is a fragmentary, partially cross-sectional, perspective view of a collapsed occluder set 117 within a delivery tube 108 in cross-section.

FIG. 15 is a fragmentary, partially cross-sectional, perspective view of the occluder set 119 and delivery system of FIG. 12.

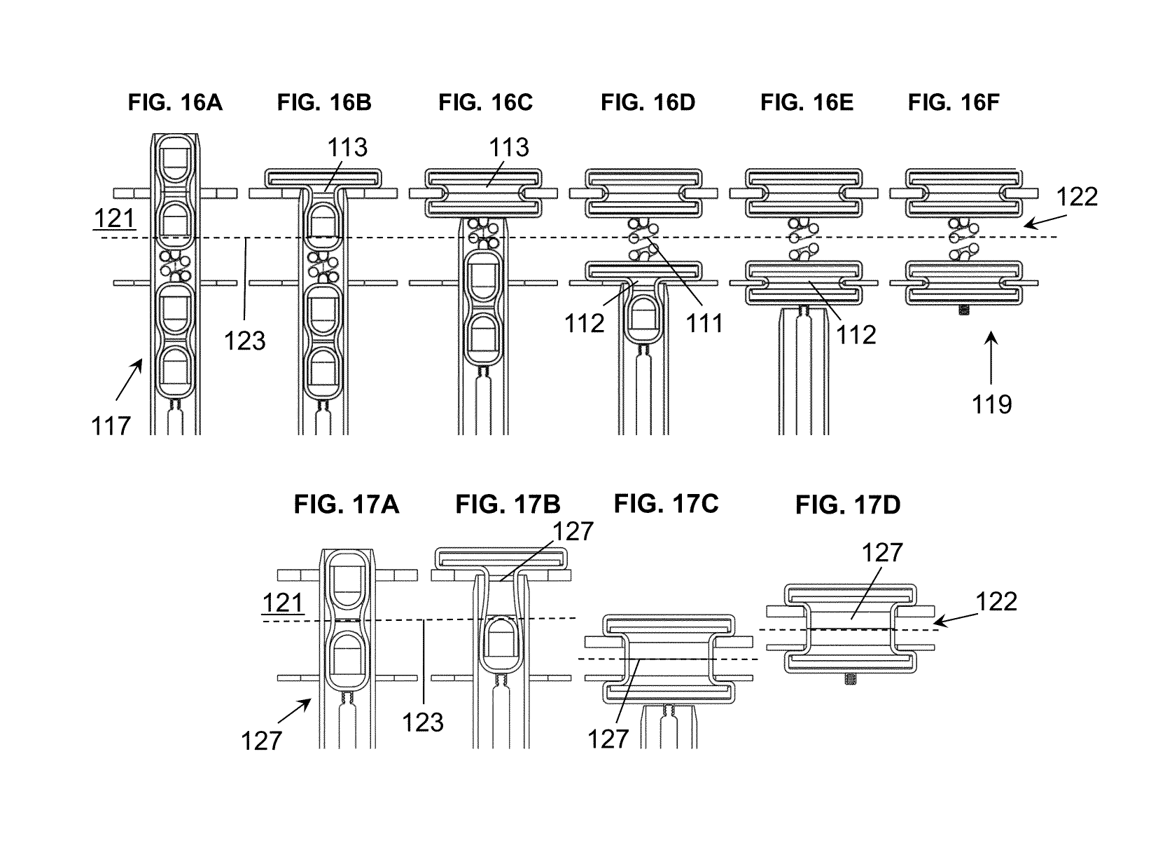

FIGS. 16A through 16F are fragmentary, side cross-sectional, diagrammatic views of a sequential implantation of an occluder set into apertures located within partial vessel walls. A pre-implantation interstitial gap width 121 and a post-implantation interstitial gap width 122 are shown to exhibit the lack of relative vessel displacement the herein-described systems and methods exhibit due to the lack of system tensions. The interstitial gap 121 is also defined by a central axis 123. Stages of the sequential deployment are detailed as follows:

FIG. 16A—insertion of the collapsed occluder set 117 through apertures;

FIG. 16B—partial expansion of the distal occluder 113;

FIG. 16C—full expansion of the distal occluder 113;

FIG. 16D—partial expansion of the proximal occluder 112 with an expanded tether 111;

FIG. 16E—full expansion of the proximal occluder 112; and

FIG. 16F—an implantation of the occluder set 119.

No change of the interstitial gap 121 or the central axis 123 is shown. The pre-implantation interstitial gap width 121, the post-implantation interstitial gap width 122, and the central axis 123 remain the same throughout operation of the occluder set 119. Discrete stage instructions from the delivery system can be used to more precisely implant occluders.

FIGS. 17A to 17D are fragmentary, side cross-sectional, diagrammatic views similar to FIGS. 16A to 16F and diagrammatically represent sequential implantation of a prior art relative vessel tension-based occluder into apertures located within partial vessel walls. The pre-implantation interstitial gap width 121 and the post implantation interstitial gap width 122 are shown and exhibit changes caused by the relative vessel tensions that prior art devices exhibit. The interstitial gap 121 is also defined by the central axis 123, which is displaced during implantation. Stages of sequential deployment are detailed as follows:

FIG. 17A—collapsed prior art occluder 127;

FIG. 17B—partially expanded prior art occluder 127;

FIG. 17C—fully expanded prior art occluder 127; and

FIG. 17D—an implanted prior art occluder 127.

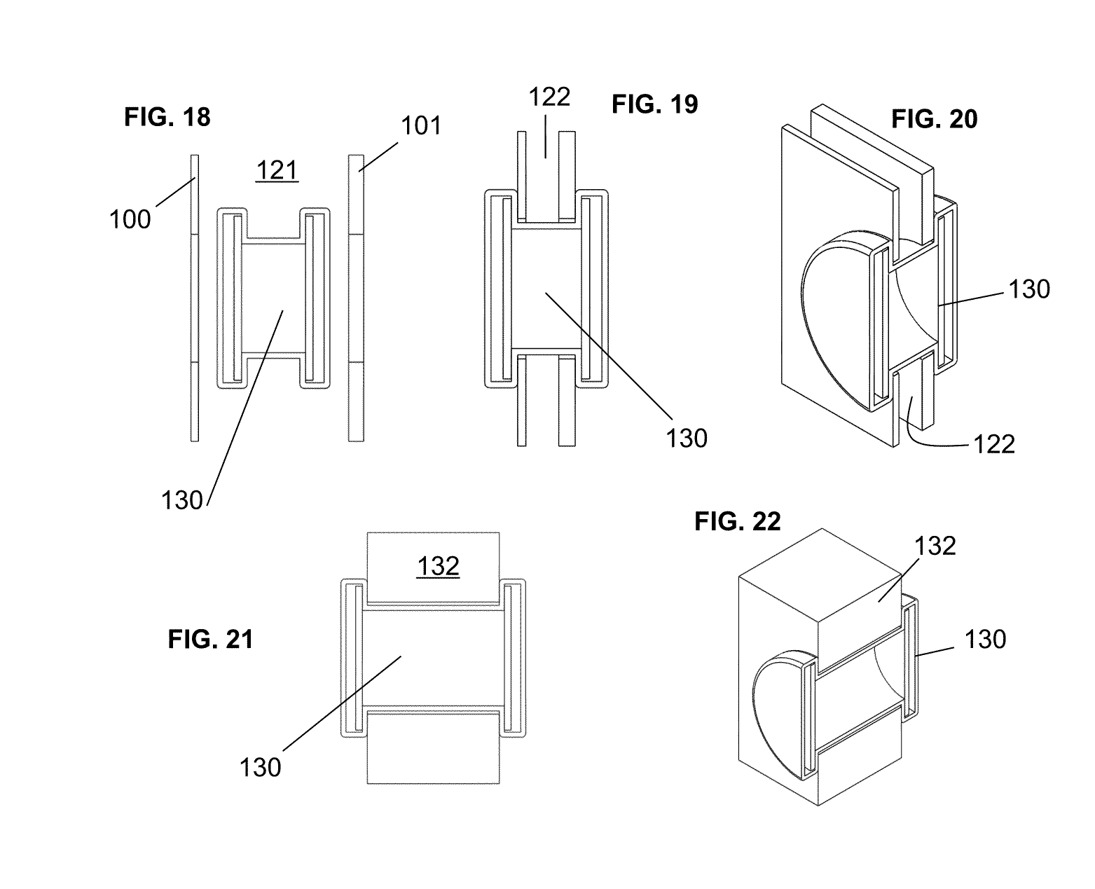

FIG. 18 is a fragmentary, side cross-sectional, diagrammatic view of a nominal prior art occluder 130 superimposed into an interstitial space 121 between a scaled representation of vessel walls 100, 101. As above, the pre-interstitial gap is shown with reference numeral 121.

FIG. 19 is a fragmentary, side cross-sectional, diagrammatic view of an implanted prior art occluder 130 into a scaled representation of the vessel walls. Similar to FIG. 17, the post interstitial gap 122 is shown to be different and smaller than the pre-interstitial gap of FIG. 18. FIG. 20 is a fragmentary, cross-sectional, perspective view of the view of FIG. 19.

FIG. 21 is a fragmentary, side cross-sectional, diagrammatic view of the implanted occluder 130 into a single wall thickness aperture 132. Prior art occluders are designed specifically for this condition. FIG. 22 is a fragmentary, cross-sectional, perspective view of FIG. 21.

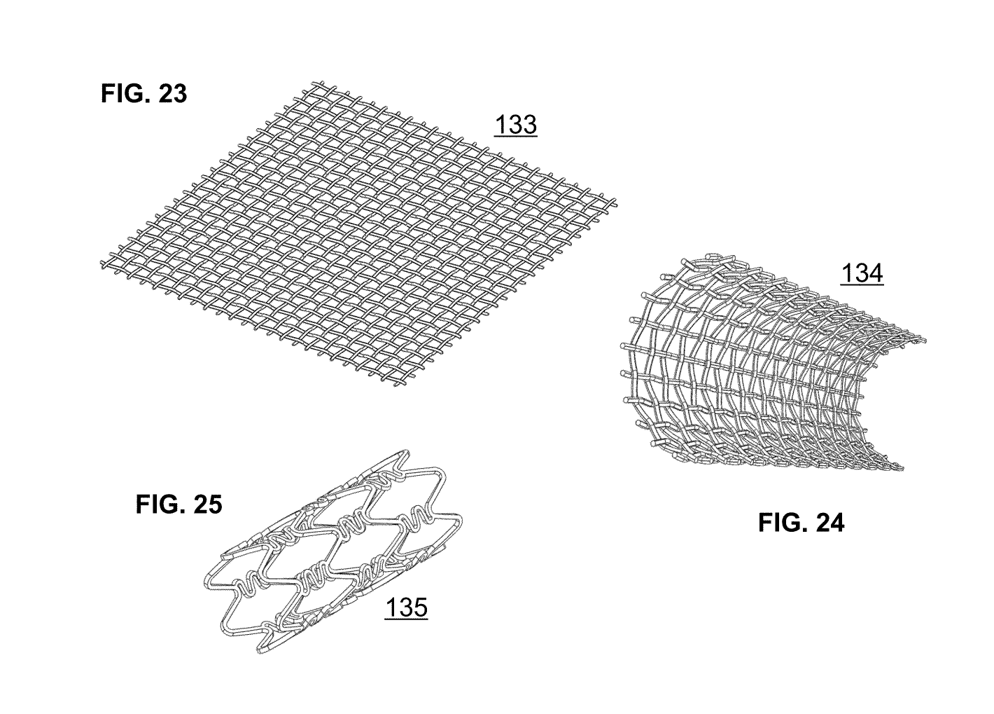

FIG. 23 is a fragmentary, perspective view of a woven mesh 133. FIG. 24 is a fragmentary, perspective view of a sectioned portion of a tubular woven mesh 134. Occluders may comprise such a tubular woven mesh.

FIG. 25 is a fragmentary, perspective view of a tubular machined structure 135. Occluders may comprise such a tubular machined structure.

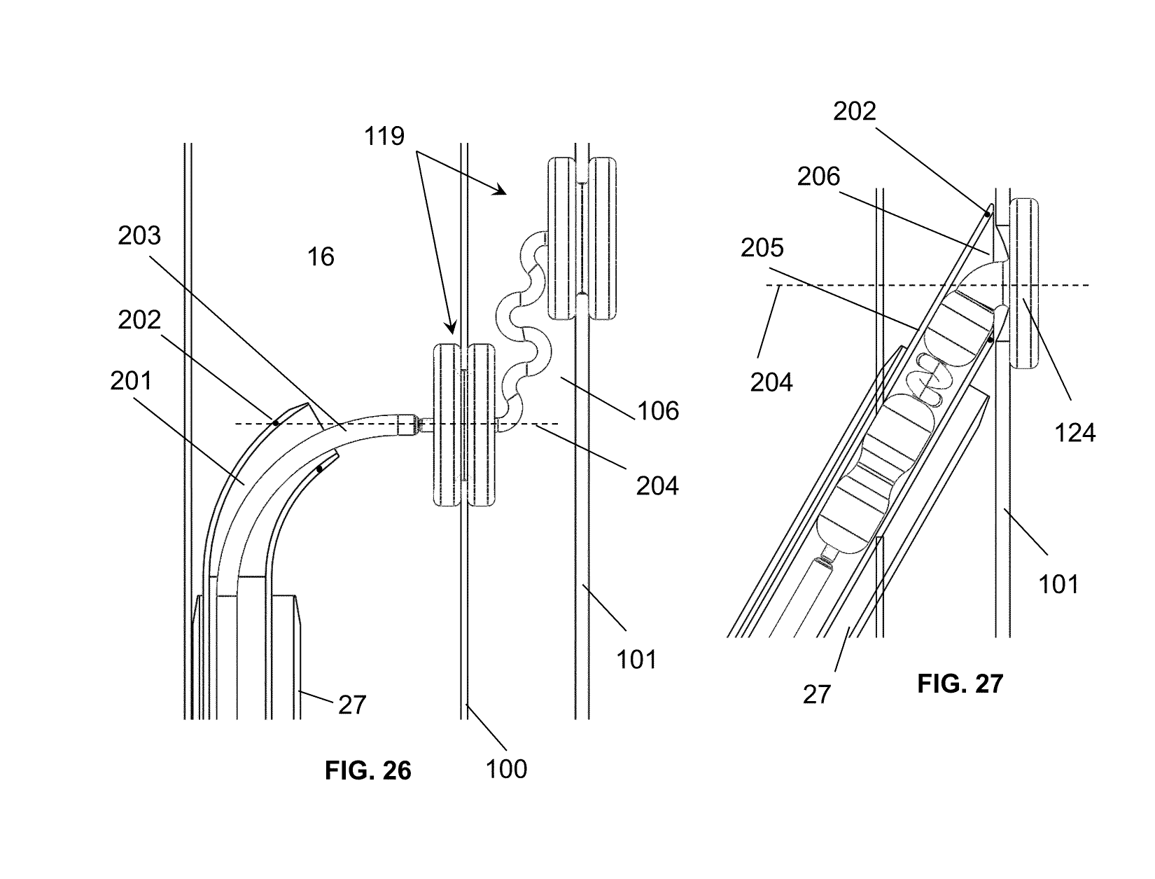

FIG. 26 is a fragmentary, side partially cross-sectional, diagrammatic view of the occluder set 119 fully expanded into the vessels walls 100, 101. The occluder set 119 is attached to a curved delivery member 203 and a curved delivery tube 201. The delivery system is shown within an introducer sheath 27. The overall geometries of the curved delivery system allow for an implantation axis that is parallel to the central aperture axis 204. Radiopaque markers 202 are included for correct orientation reference during fluoroscopic guidance. The ends of the expanded tether 106 are shown offset in a vertically displaced orientation.

FIG. 27 is a fragmentary, side partial cross-sectional, diagrammatic view of a distal occluder 124 in a partially expanded state in vessel wall 101. The occluder 124 is shown as being delivered by a delivery tube 205 having an obliquely angled cut distal section 206 with an opening axis parallel to the central aperture axis 204. Radiopaque markers 202 are included for correct orientational reference during fluoroscopic guidance.

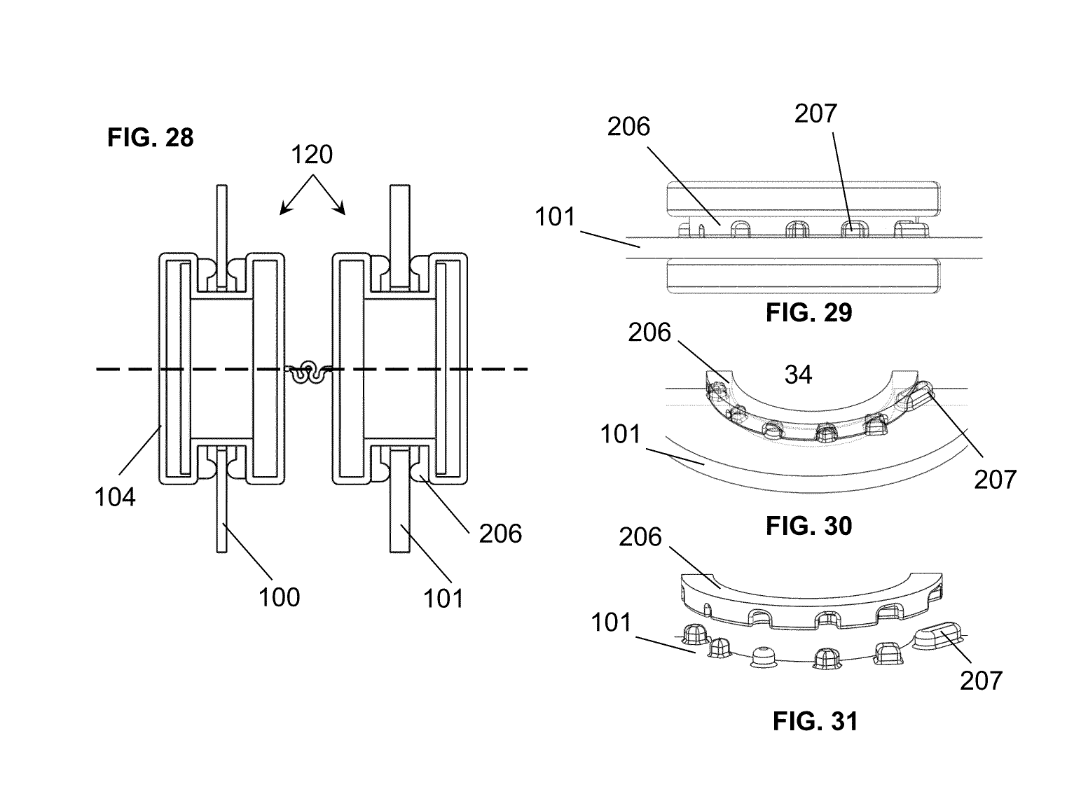

FIG. 28 is a fragmentary, side cross-sectional, diagrammatic view of an implanted occluder 120 into vessel walls 100, 101. The occluders 120 feature sealing skirts 206 external of the occluder frame 104. The sealing skirts 206 have a beaded section (or spaced protuberances about its periphery) to increase localized compliance around the area of the aperture's perimeter.

FIG. 29 is a fragmentary, diagrammatic side view of a single implanted occluder into a vessel wall 101. The occluder features a sealing skirt 206 on one side. The compliant sealing skirt 206 is shown as conforming to surface irregularities 207 of the vessel wall 101. These irregularities represent the presence of calcium, atherosclerotic media, and vessel thickening, for example.

FIG. 30 is a fragmentary, diagrammatic frontal, angled view of a section of the vessel wall 101 and a transparent isolated sealing skirt 206. Surface irregularities 207 are shown to be mostly encapsulated by the sealing skirt 206 and contact of the implant around the aperture 34 is demonstrated. FIG. 31 is similar to FIG. 30 but shows a conforming and separated sealing skirt 206 for clarity.

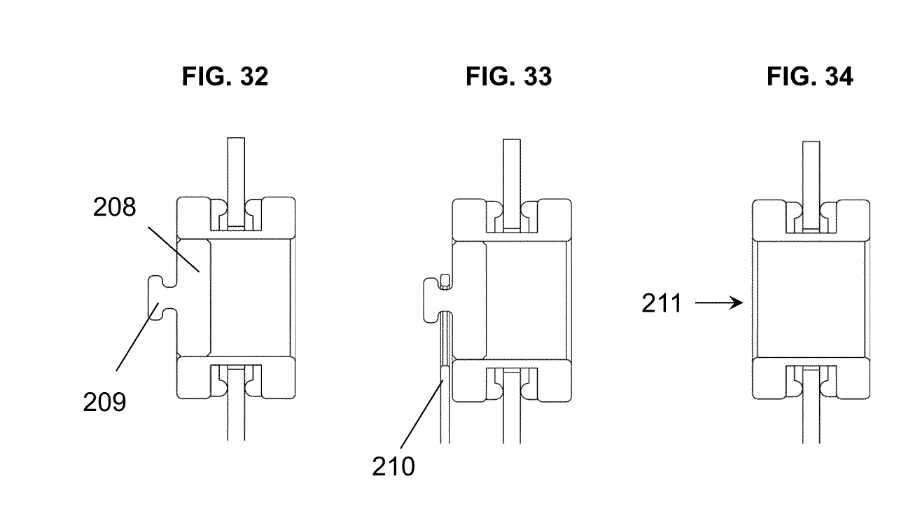

FIG. 32 is a fragmentary, cross-sectional, diagrammatic, side view of a single implanted occluder similar to the one shown in FIG. 28. The occluder has an onboard central reentry plug 208, which plug 208 has a reentry connection feature 209 used during initial implantation of the occluder as well as for future removal of the occluder plug 208. FIG. 33 is a fragmentary, cross-sectional, diagrammatic, side view similar to FIG. 32 and includes a reentry plug member 210 engaged on a reentry connection feature 209 of the reentry plug 208. FIG. 34 is a fragmentary, cross-sectional, diagrammatic, side view similar to FIG. 32 and shows the reentry plug 208 removed, thereby creating a central occluder path 211.

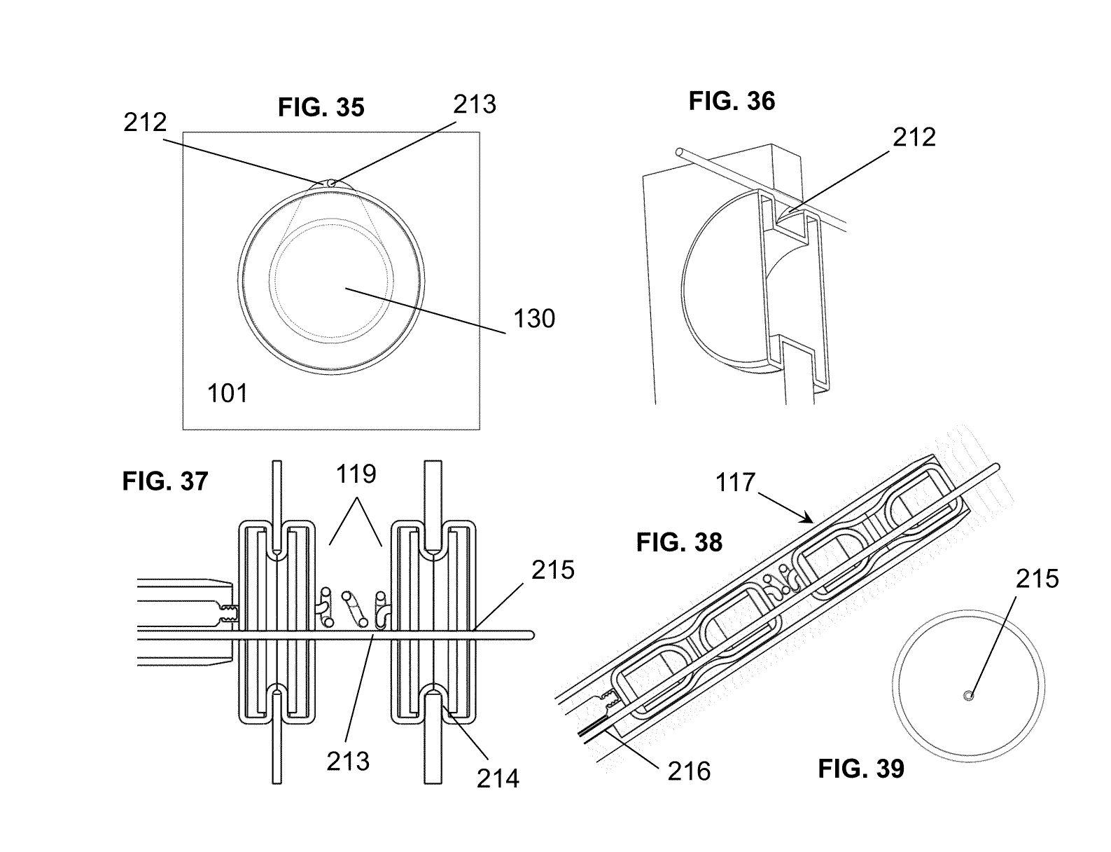

FIG. 35 is a fragmentary, front view of an implanted prior art occluder 130 into a vessel wall 101 with a parallel-to-axis guidewire 213. As can be seen, the parallel-to-axis guidewire 213 interrupts the sealing contact surface of the occluder 130 and, as a result, creates a leak path 212. FIG. 36 is a fragmentary, cross-sectional, perspective view of FIG. 35 with the leak path 212 observed.

FIG. 37 is a fragmentary, side cross-sectional, diagrammatic view of an implanted and attached occluder set 119 into vessel walls. The occluder set 119 includes a guidewire 213 located within the occluder set 119 and on the same cross-sectional plane. A guidewire path 215 allows for central guidewire pass-through. Contrary to FIGS. 35 and 36, the inventive occluder set 119 demonstrates full contact of the perimeter 214 of the aperture.

FIG. 38 is a fragmentary, side cross-sectional, diagrammatic view of a collapsed occluder set 117 with a central to delivery system guidewire. A physical guidewire lumen 216 is shown attached to delivery tube. A similar feature, such as the lumen 216, can be used for rotational keying and aligning features to maintain correct relative relationships with alignment markers during loading of the occluders 117 into the delivery tube.

FIG. 39 is a fragmentary, frontal view of a single occluder with a central guidewire path 215.

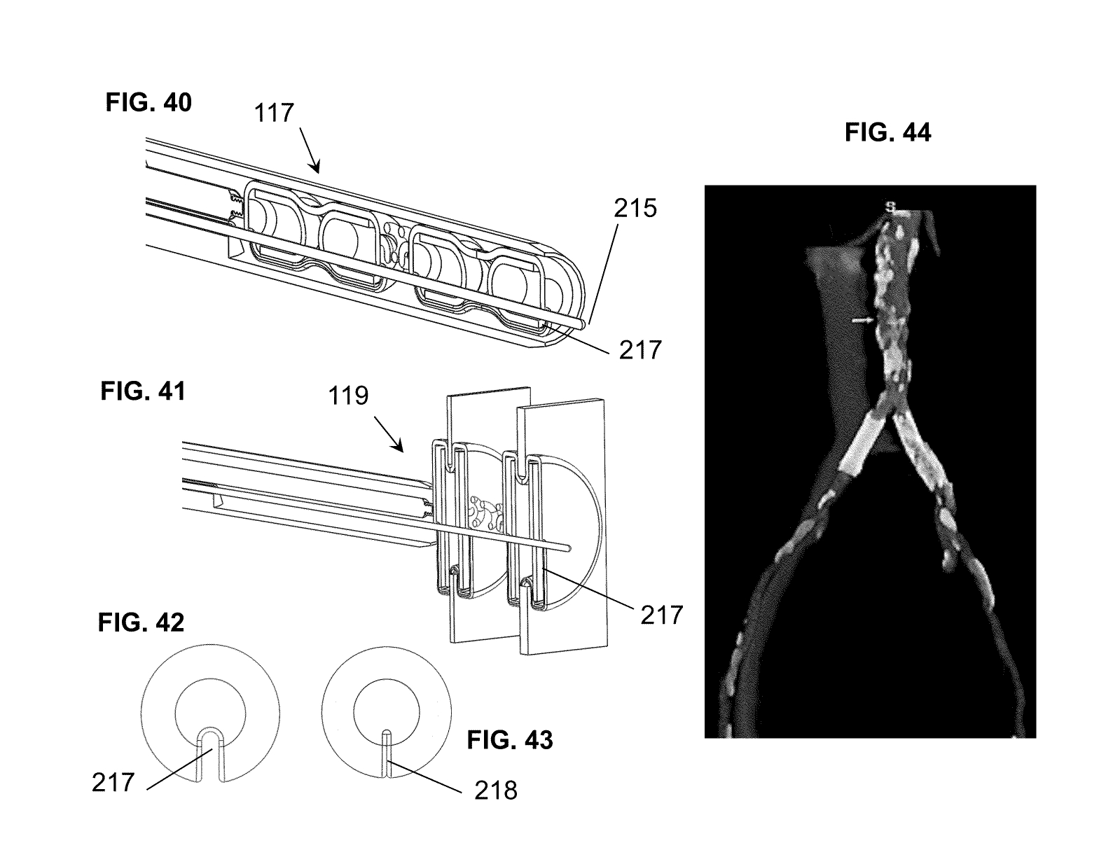

FIG. 40 is a fragmentary, cross-sectional, perspective view of a collapsed occluder set 117 with an open guidewire channel 217 and a loaded guidewire 215.

FIG. 41 is a fragmentary, cross-sectional, perspective view similar to FIG. 40 but includes an expanded and attached occluder set 119 with an open guidewire channel 217.

FIG. 42 is a fragmentary, frontal view of a single collapsed occluder with an open guidewire channel 217.

FIG. 43 is a fragmentary, frontal view similar to FIG. 42 but with a collapsed occluder with a closed guidewire channel 218.

FIG. 44 is an anterior CT image of severely diseased aortic vessels. Highlighted areas represent presence of calcium and atherosclerotic plaque. An arrow identifies a possible transcaval access path.

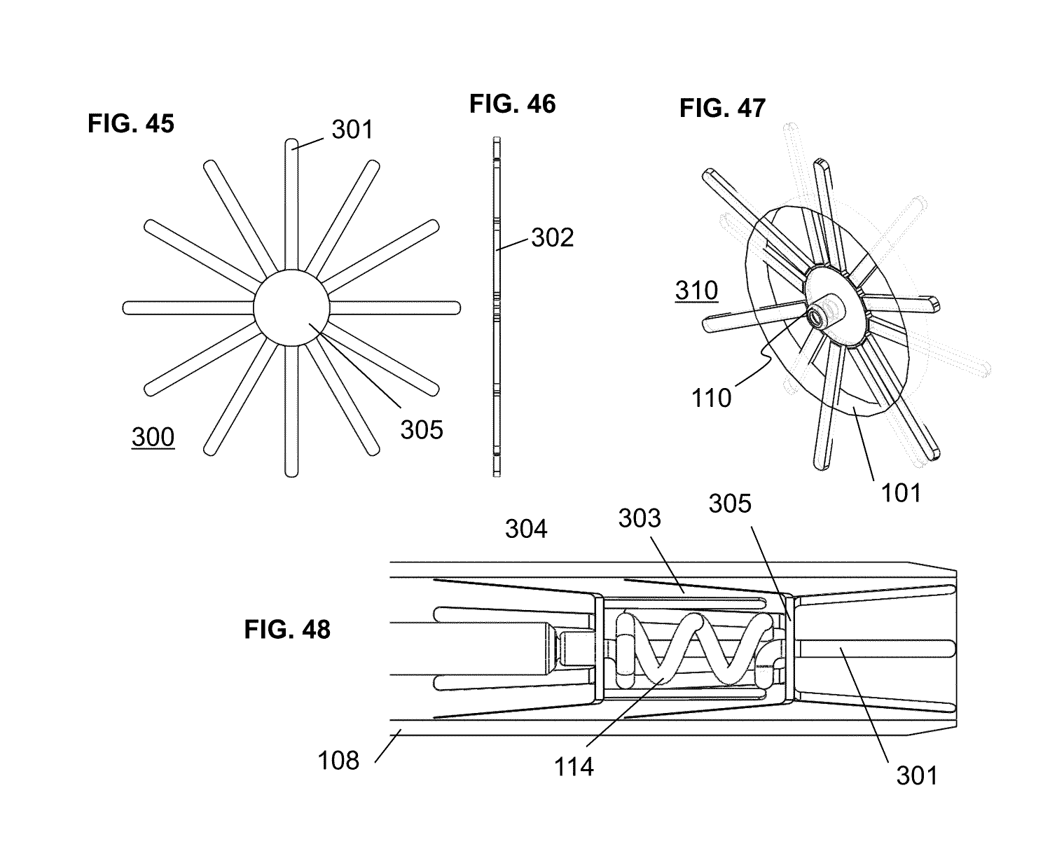

FIG. 45 is a fragmentary, frontal view of an occluder flat beam frame 300 having a radial array of beams 301 that define a generally circular outer perimeter. FIG. 46 is a fragmentary, side view of the occluder frame 300 from FIG. 45 and demonstrates a generally flat structure having a wall thickness 302.

FIG. 47 is a fragmentary, perspective and semi-transparent view of an implanted flat beam occluder 310 within a vessel wall aperture 101. The beam array is shown in an alternating fashion and is defined by opposing groups of beams relative to the vessel wall. Elastic properties of the frame provide attachment forces to the vessel wall. A connection member 110 is shown.

FIG. 48 is a fragmentary, cross-sectional, side view of a collapsed flat frame occluder set 304 connected by a tether 114 and housed within a deployment tube 108. The central section 305 of the occluder frame is generally concentric with the deployment tube 108. The beam array 301 is restrained in an alternating configuration that creates groups of opposing beams relative to a hub or central section 305. The distal beams of the proximal occluder frame and the proximal beams of the distal occluder are held in an interleaved configuration 303.

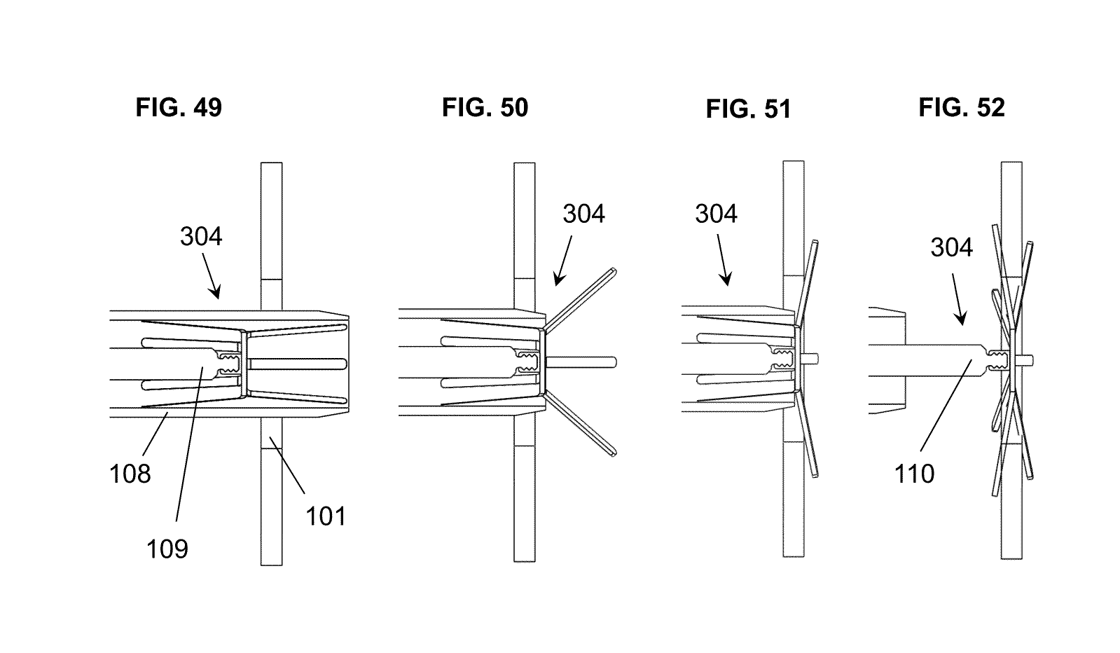

FIG. 49 is a fragmentary, cross-sectional, side view of a single collapsed flat frame occluder 304 within a vessel wall aperture 101. FIG. 50 is a fragmentary, cross-sectional, side view of the single partially expanded flat frame occluder 304 during a transition between its collapsed state and partially expanded contact state within the vessel wall aperture 101. FIG. 51 is a fragmentary, cross-sectional, side view of the single partially expanded flat frame occluder 304 within the vessel wall aperture 101. FIG. 52 is a fragmentary, cross-sectional, side view of a single expanded flat frame occluder 304 within the vessel wall aperture 101 just before detachment of the connection member 110.

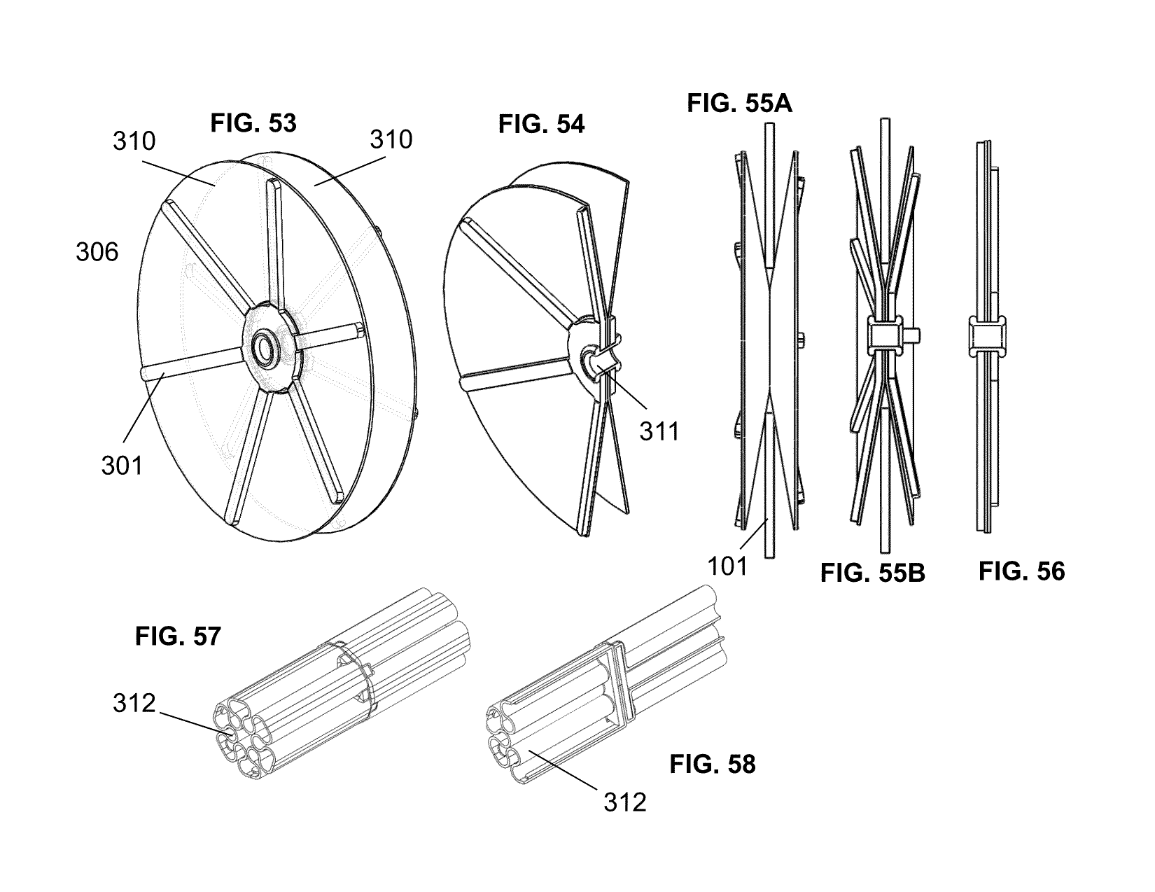

FIG. 53 is a fragmentary, semi-transparent, perspective view of a laminated flat frame occluder 306 in a fully expanded state. The occluder 306 includes independent flat beam arrays 301 opposing two sealing member sheets 310. The beam arrays 301 are shown in a rotationally indexed configuration. FIG. 54 is a fragmentary, cross-sectional view of the occluder 306 in FIG. 53 and includes a laminated assembly retention member 311 that retains the laminated structure as well as an optional central hub member or guidewire path. The retention member also can be made as an extension of a tether member. FIG. 55A is a side elevational view of the occluder 306 deployed into a vessel wall aperture 101. FIG. 55B is a cross-sectional view of the occluder of FIG. 53 positioned within the vessel wall aperture 101. FIG. 56 is a cross-sectional view of the occluder 306, shown in a nominal expanded state without a vessel wall located in between sealing materials of the occluder that demonstrates a sequentially contacting laminated assembly with no preset vessel wall thickness gap.

FIG. 57 is a fragmentary, perspective view of a collapsed form of the flat beam occluder 304 and demonstrates the sealing member in a collapsed pleated configuration 312 that resides within a general minimum diameter. FIG. 58 is a cross-sectional view of the occluder 304 of FIG. 57.

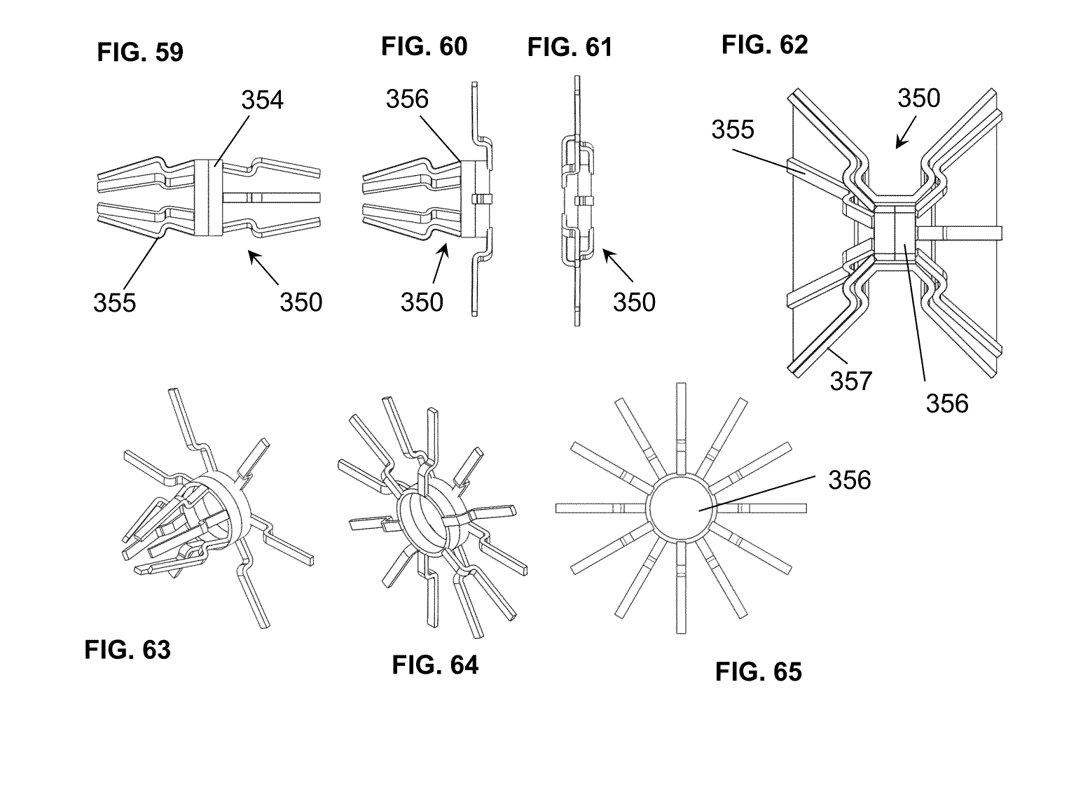

FIG. 59 is a side view of a collapsed tubular beam occluder 350 and is similar to a machined stent. This occluder 350 can be manufactured from a tube and then formed. Tubular occluder beams 355 are shown formed. These beams 355 actuate with respect to a central section 354, as shown in FIGS. 60 and 61. In FIG. 60, the tubular beam occluder 350 is partially expanded and the beams 355 actuate with respect to the central section 354 at bend locations 356. Finally, FIG. 61 shows the tubular beam occluder 350 in a fully expanded state with the beams 355 formed to achieve a minimum or negative central clamping gap.

FIG. 62 is a cross-sectional, side view of an entirety of the tubular beam occluder 350 including a sealing member 357 attached to frame that is continuous about the central axis of the occluder. FIGS. 59 to 61 and 63 to 65 shows the occluder 350 symmetrically transitioning between its collapsed state (FIG. 59) and its fully expanded state (FIGS. 60, 61, 65, and 65). FIG. 65 shows the front view of occluder, which demonstrates an open path 356 within the occluder frame's center section.

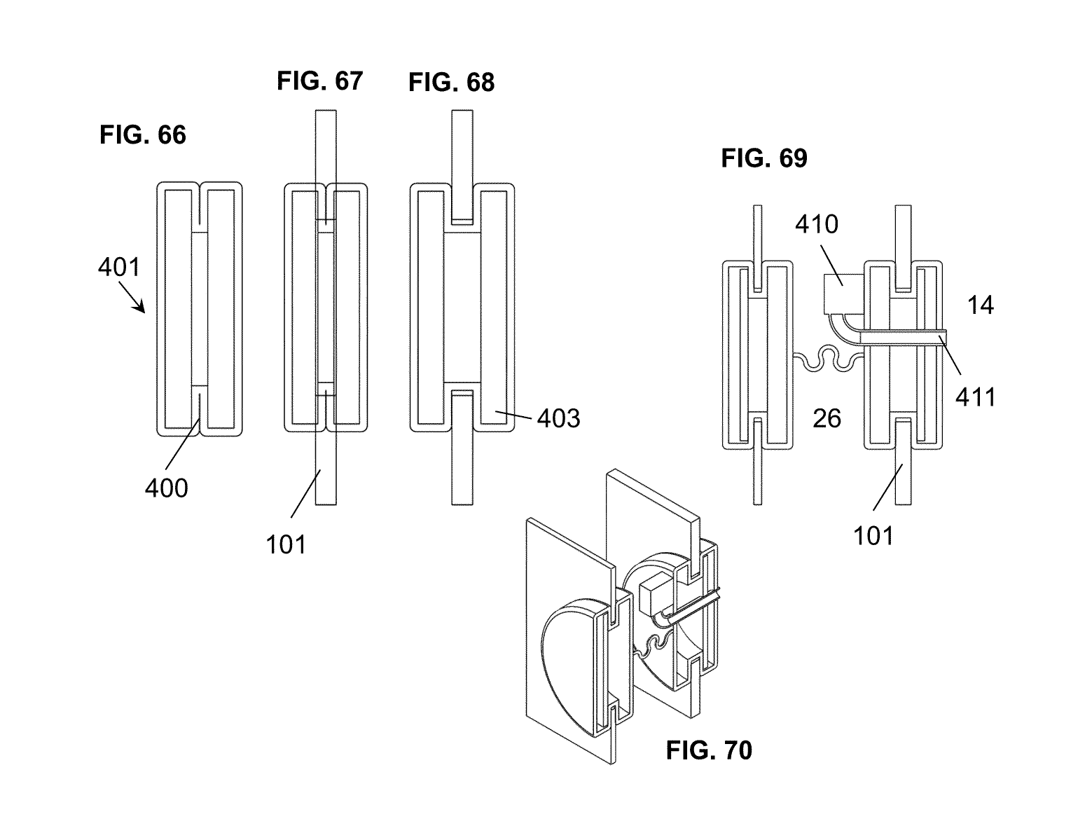

FIG. 66 is a cross-sectional side view of a single occluder 401 with a zero-waist length in its nominal position. The waist location 400 is shown as the center section of the occluder structure and is a smaller diameter than the outer disks 403 of the occluder 401 and fits within an aperture diameter. FIG. 67 is a fragmentary, cross-sectional side view of the single zero-waist length occluder 401 superimposed onto a vessel wall aperture 101. FIG. 68 is a fragmentary, cross-sectional side view of the single implanted zero-waist length occluder 401. Here, however, the outer disks 403 of the occluder 401 are stretched across the vessel wall.

FIGS. 69 and 70 show an implanted occluder set with a sensor 410 residing in the interstitial space 26. The sensor 410 has a conduit 411 that creates a blood path from the inside of the vessel to the sensor 410.

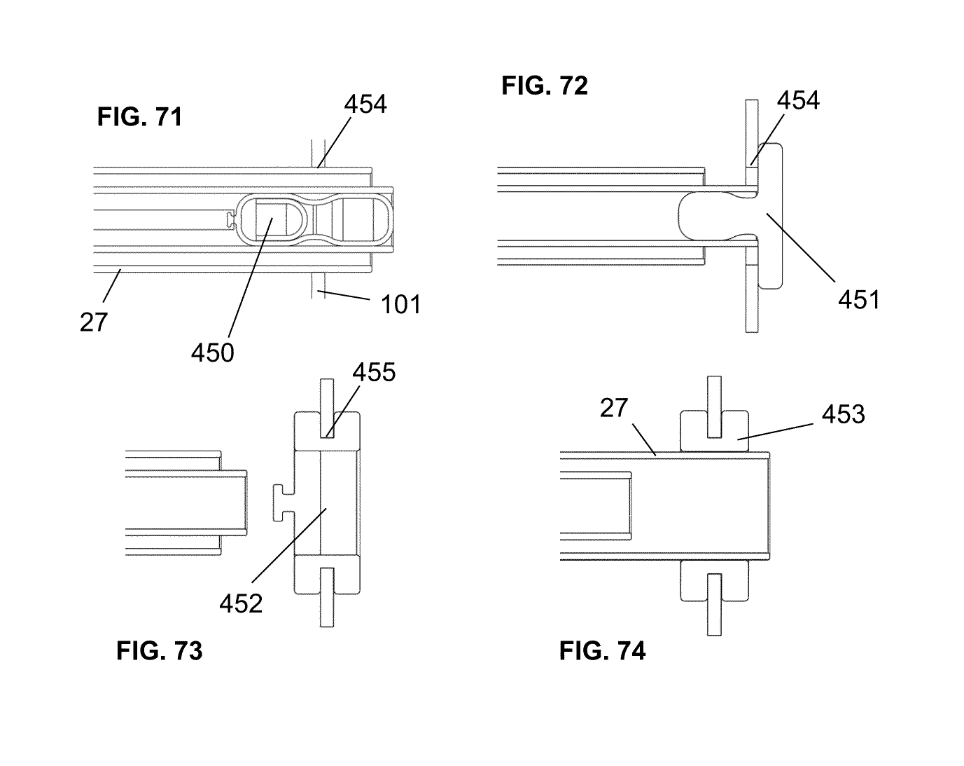

FIG. 71 is a fragmentary, cross-sectional, side view of a single collapsed occluder with a sheath reentry port 450 within an introducer sheath 27 inserted into a vessel wall aperture 101 having a starting diameter 454. FIG. 72 shows a single occluder partially expanded with a sheath reentry port 451. FIG. 73 is a fragmentary, cross-sectional, side view of a single implanted occluder with a sheath reentry port 452. Implantation of the occluder dilates the vessel aperture from the starting diameter 454 to an implantable diameter 455. FIG. 74 is a fragmentary, cross-sectional, side view of a single implanted occluder with a reentry plug removed and the introducer sheath 27 from FIG. 71 passing through the central port of the occluder.

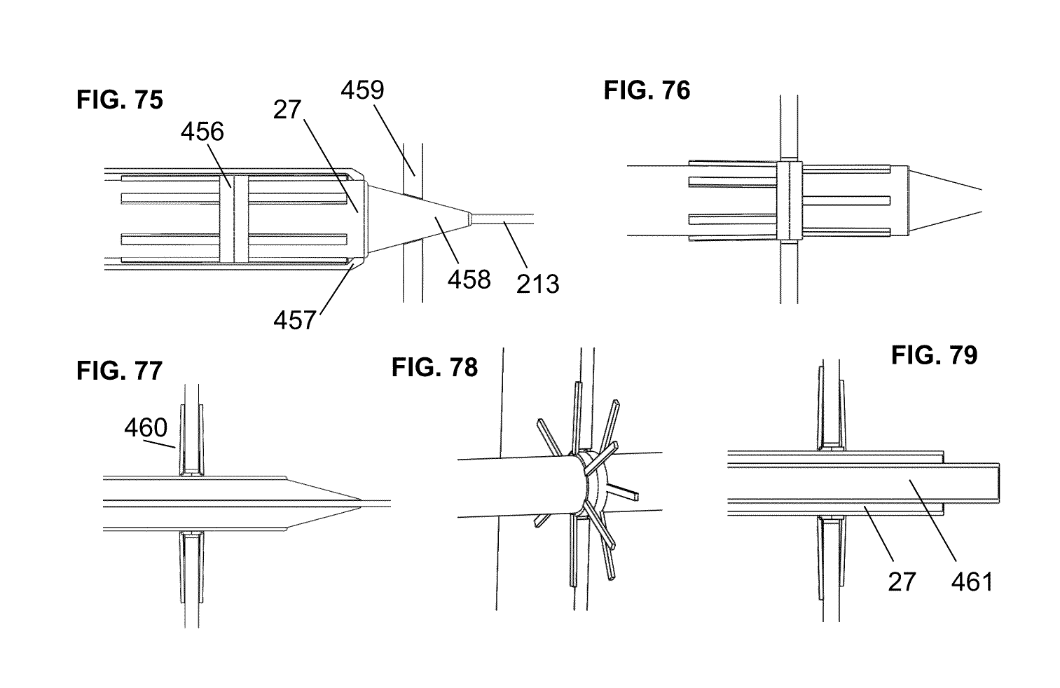

FIG. 75 is a fragmentary, cross-sectional, side view of a collapsed introducer sheath with an occluder 456 similar to 350 loaded therein. The occluder 456 is loaded onto the introducer sheath 27 and is concealed by a sheath outer tube 457. An introducer sheath dilator 458 is shown as creating a vessel wall aperture 459. The assembly is shown in FIG. 75 with a central guidewire 213. FIG. 76 is a fragmentary, cross-sectional, side view of the occluder 456 from FIG. 75 in a correct implantation location central to the vessel wall.

FIG. 77 is a fragmentary, cross-sectional, side view of an expanded, introducer sheath loaded occluder 460 and FIG. 78 shows the occluder detailed in FIG. 77 from an angle to the vessel wall.

FIG. 79 is a fragmentary, cross-sectional, side view of an expanded introducer sheath loaded occluder with an introducer sheath 27 through central occluder section including a general catheter device 461.

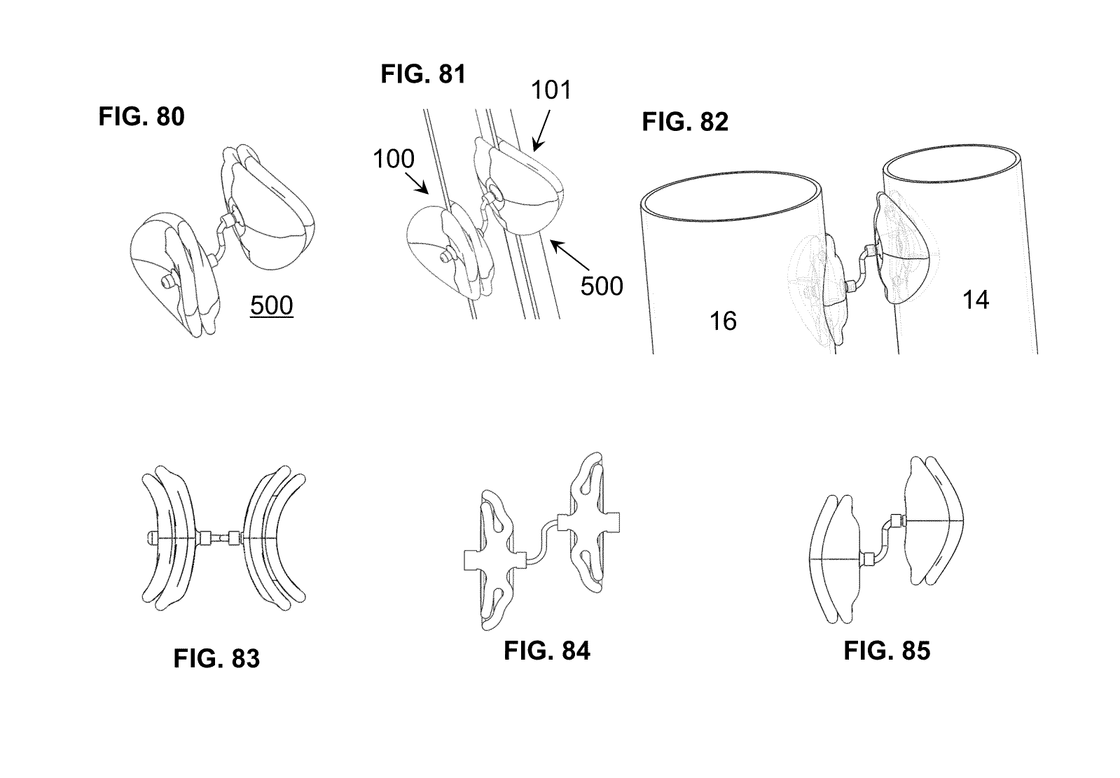

FIG. 80 is a perspective view of a non-circular occluder set 500 in a nominal shape. FIG. 81 is a fragmentary, partially cross-sectioned, perspective view of an implanted non-circular occluder set 500 within vessel apertures 100 and 101. FIG. 82 is a fragmentary, perspective view of an implanted non-circular occluder set 500 within vessels 16 and 14. FIG. 83 is a fragmentary, top view of the occluder set 500 showing an arc geometry having a center parallel with a vessel's center axis. FIG. 84 is a cross-sectional view of the occluder set 500. FIG. 85 is a side view of occluder set 500 that demonstrates generally linear geometries that match vessel geometries and are different to geometries shown in top view.

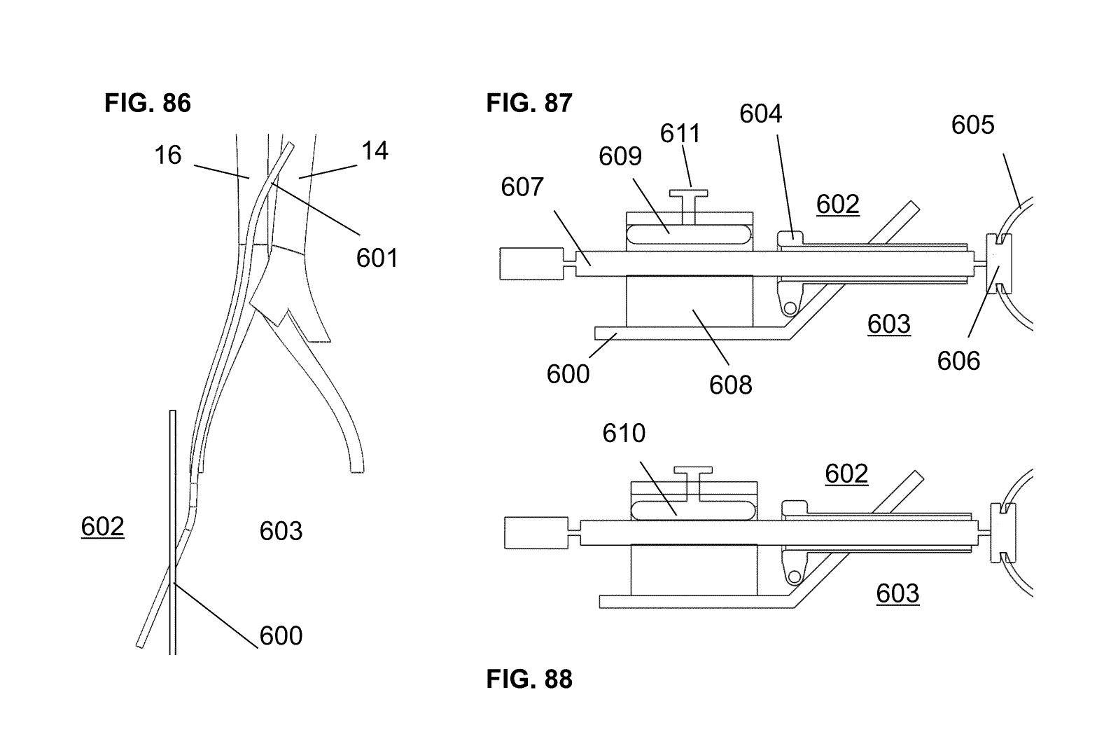

FIG. 86 is a fragmentary illustration of a human aortic 14 and venous 16 vascular system with a superimposed conduit 601 demonstrating a path from outside of the body 602 through a skin surface 600 into a femoral vein 16 through a transcaval access, and into the aorta 14.

FIG. 87 is a fragmentary, diagrammatic side view of a deployment system for an occluder 606 including the vessel 605 in which the occluder is to be deployed, the inside of the body 603, the skin surface 600, an introducer sheath 604, the outside of the body 602, an occluder delivery system 607, an anti-pull out lock housing 608, a disengaged anti-pull out lock 609, and an anti-pull out lock plunger 111. The anti-pull out lock 609 is shown in the disengaged position. In FIG. 88, the anti-pull out lock is in its engaged position 610.

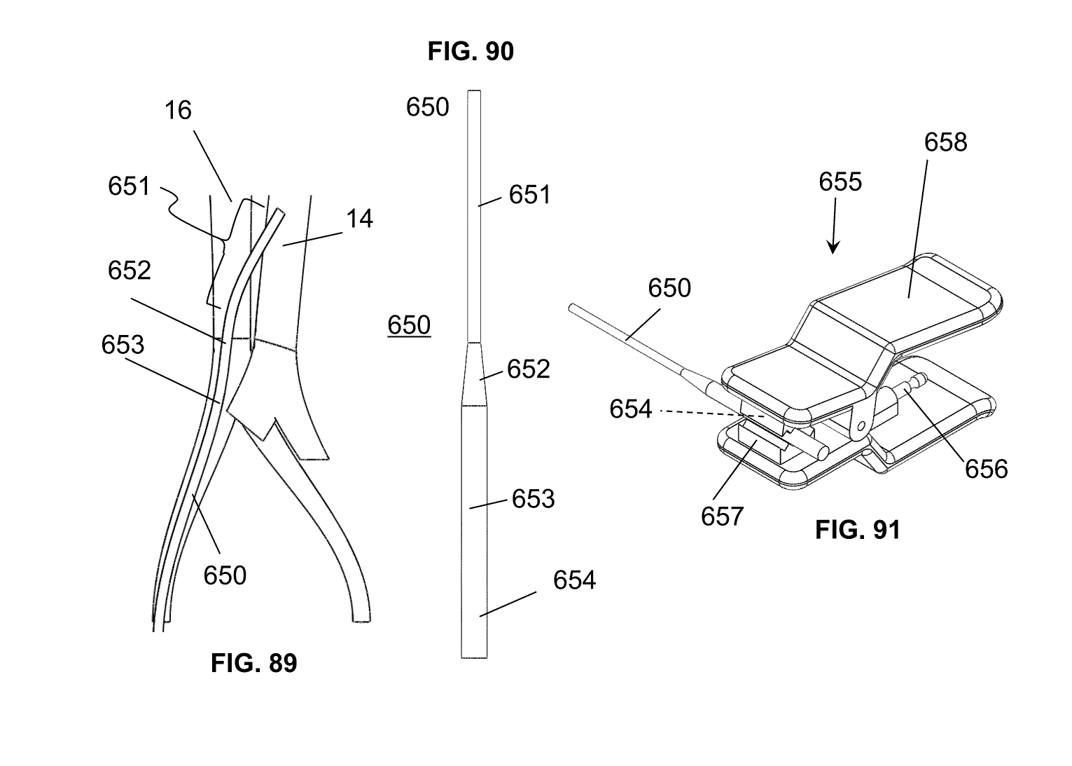

FIG. 89 is a fragmentary illustration of a human aortic 14 and venous 16 vascular system with a superimposed performance guidewire 650 that includes a larger diameter proximal section 653, a smaller diameter distal section 651, and a transition section 652. The variable diameters throughout the length of the performance guidewire 650 create stiffer and less stiff sections that facilitate improved articulation to conform to anatomy and improved manipulation throughout introduction, vessel punctures, advancement into vasculature and closure during access procedure. The performance guidewire 650 is manipulated such that smaller diameter and less stiff section resides around the closure area to reduce the amount of forces transferred to the occluders for a more accurate and unobstructed placement and seal assessment that is similar to a fully implanted occluders. FIG. 90 is a fragmentary, top view of the performance guidewire 650. In another embodiment, variable stiffness zones as previously described can be achieved using various material, coiled, braided, or cabled wire sections to yield variable stiffness's while maintaining constant diameters. FIG. 91 is a fragmentary, perspective view of an electrocautery guidewire adapter 655 attached to the performance guidewire 650. The guidewire adapter 655 has a standard cautery electrical connector 656, an atraumatic guidewire clamp 657, and a hand-operated actuation device 658. An electrical connection is transferred to the cautery connector through the guidewire clamp and into a conductive section 654 of the performance guidewire 650. In another embodiment, guidewire clamp 657 and electrically conductive section 654 may translate and/or rotate with respect to the guidewire adapter 655 to reduce load onto guidewire during manipulation. Guidewire adapter 655 can be compatible with standard guidewires. In an additional embodiment, guidewire adapter 655 may perform equivalent to a standard guidewire clamp handle and include a similar pin vice style clamp in order to advance and manipulate guidewire through anatomy.

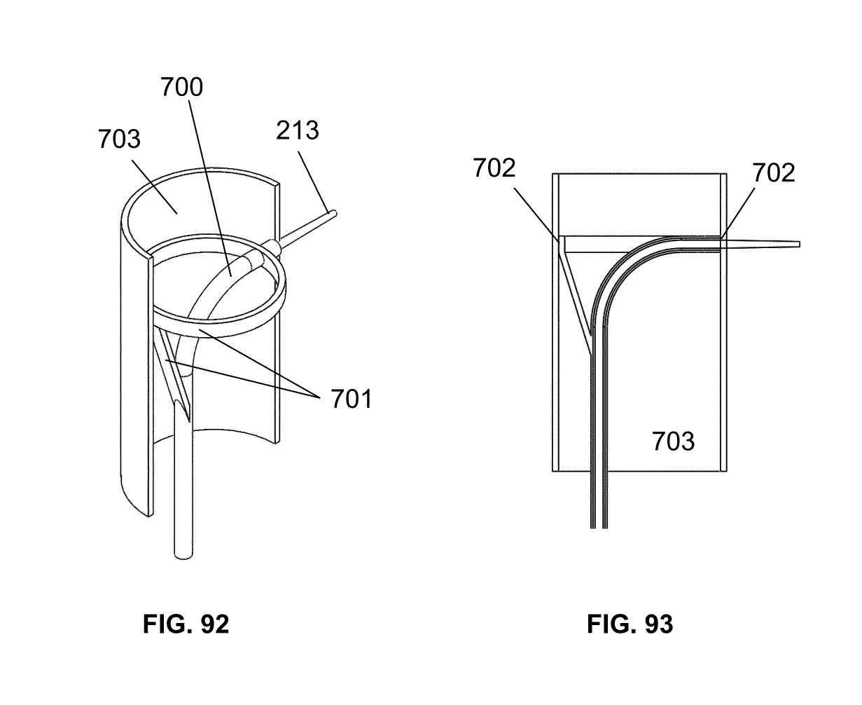

FIG. 92 is a fragmentary, partially cross-sectional, perspective view of a guide catheter 700 with support members 701 within a vessel 703. A guidewire 213 is shown exiting the guide catheter 700 and extending through the wall of the vessel 703. FIG. 93 is a fragmentary, cross-sectional, side view of the diagram of FIG. 92 and demonstrates multiple catheter-to-vessel contact points 702.

In greater detail, a flat beam frame occluder shown in FIGS. 45 to 58 can be defined as a one-layer or multi-layer elastic spring material with a radial beam array that defines an outer diameter as well as a central section. Similarly, a tubular structure can be machined and formed to create a tubular beam frame. An impermeable member is attached to the structure to establish a sealing curtain across the outer diameter central surface area. Beam arrays are interdigitated and correspond to opposing sides with sealing members to correspond to both sides. Beam sets are flexed away from each other to form a collapsed state. The structure in its collapsed state is loaded into a delivery tube, which constrains the structure in the collapsed state. A tether member can be attached to the central section to join two beam array occluders. The most distal occluder can be collapsed over a distal side of a proximal occluder to form an overlapped collapsed section that will be one beam length shorter than a non-overlapped set. The proximal side of the distal occluder and the distal side of the proximal occluder can be released independently or automatically. Automatic deployment is beneficial because it presents an immediate release of the external side of a venous occluder and prevent pull through. An array of double-sided beams with a nominal position about the same plane creates a zero-waist length condition and creates a contact-based auto-centering condition about the central axis of the aperture. Alternatively, the structure can have forms and features to dictate a central waist diameter. An array of spring-loaded beams backing a sealing material is advantageous when coarse surfaces are present. Isolated bending beams can compensate for large differences in wall thicknesses caused by calcium or plaque. The beam length and shape can be individually altered to define a best matching structure to vessels. A collapsed occluder structure composed of a single layer structure and a sealing member requires minimal collapsed volume and allows for a large central path for other components, such as tether and guidewire paths. In addition to a delivery tube, a flap structure can be constrained simply by a purse string at the beam ends. Beams made from flat sheets can be coined or stamped to create gradual contact surfaces towards vessel walls. Beams can be allowed to bend in uniform directions to allow for a single directional pull through in the event of removal. Both occluders and tether structures can be made from a single sheet of machined material using precision machining processes such as photo-chemical etching or laser cutting. Similarly, assemblies can be laminated and riveted or welded together. If individual layers are used for sides of the occluder then a lamination of the beam arrays and the sealing member disks can be used to create ideal conditions. The system can be packaged with a separation plate or a loading assist device. Also, the system can have features on the beams to allow for pulling apart by hand.

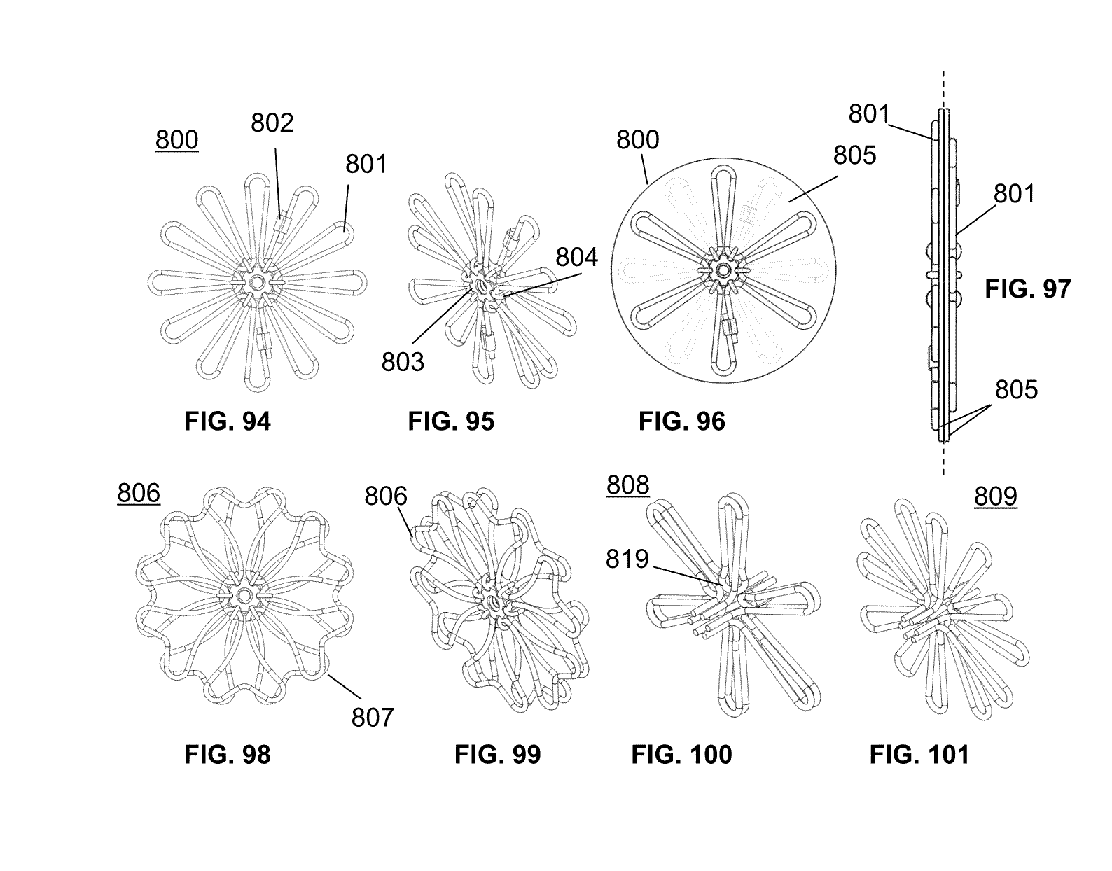

FIG. 94 is a fragmentary, frontal view of an occluder wire beam frame 800 having two groups of radial arrays of beams 801 that are each shape-set from a single wire into a petal type shape that defines a generally circular outer perimeter. Beam array groups are shown in an alternating configuration in order to distribute clamping forces between them. Both ends of the wire are approximated to form a closed loop path using a connection member demonstrated by a crimp band 802. FIG. 95 is a fragmentary, perspective view of the occluder wire beam frame 800 that demonstrates two groups of wire frames that are grounded to a central hub 803 by loops 804. FIG. 96 is a semi-transparent, frontal view of an occluder wire beam frame 800 with sealing disk 805. FIG. 97 is a side view of the occluder 800 showing two opposing groups of beam arrays 801 with sealing disks 805 about a central plane.

FIG. 98 is a fragmentary, frontal view of an occluder wire beam frame 806 that is similar to 800 and features alternating beam groups that overlap at points 807 and have a generally circumferential maximum diameter profile. FIG. 99 is a fragmentary, perspective view of an occluder wire beam frame 806.

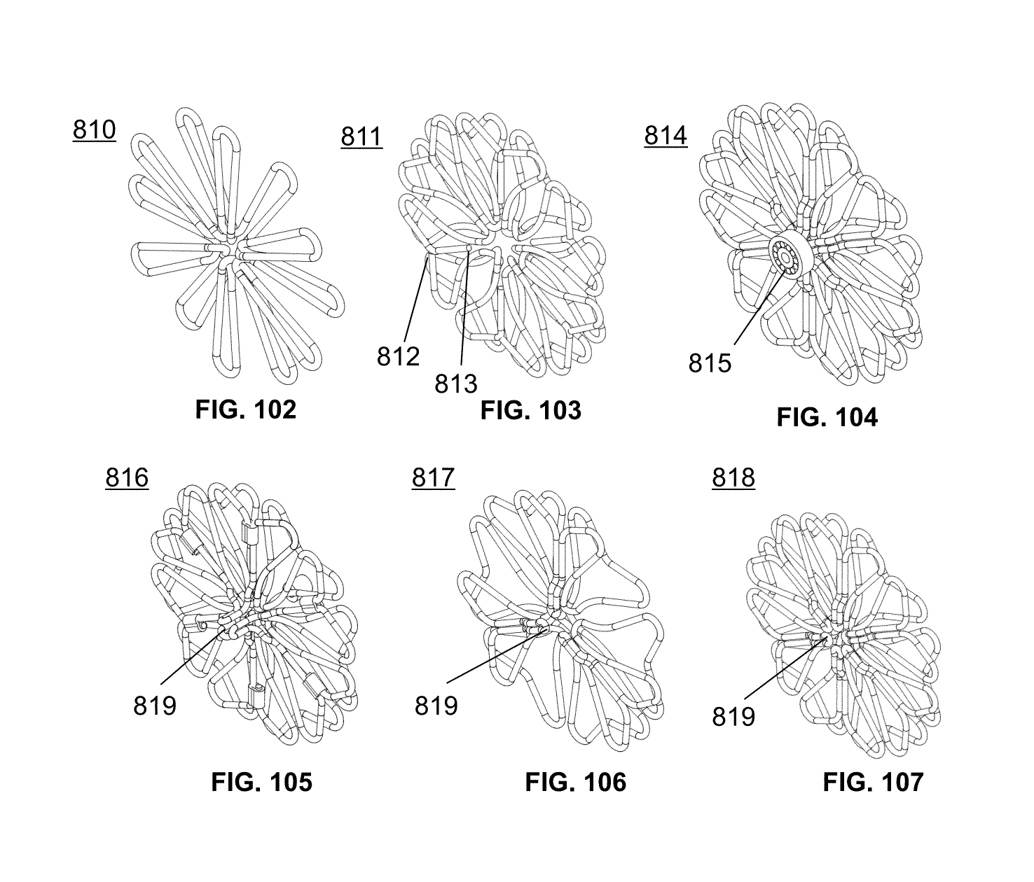

FIG. 100 is a fragmentary, perspective view of an occluder wire beam frame 808 that is composed of an array of individual wire forms that create both opposing groups of beams and does not rely on an additional central hub to provide a grounding point for beams to deflect about. Alternatively, referring to FIG. 100, wire bend transition section 819 that joins both groups of radial arrays can be positioned along (parallel to) a central axis and reside on either sides of the occluder. Wire bend transitions can also be configured to provide different levels of clamp force between the two groups. FIG. 101 is a fragmentary, perspective view of an occluder frame 809 with a radial array of alternating groups of beams formed from a single wire that do not require a central hub. FIG. 102 is a fragmentary, perspective view of an occluder wire beam frame 810 that features a radial array of alternating beam groups that is formed from a single closed loop wire, and which do not extend parallel the central axis.

FIG. 103 is a fragmentary, perspective view of an occluder wire beam frame 811 that is composed of an array of wire forms that create both overlapping opposing groups of beams and does not rely on an additional central hub to provide a grounding point for beams to deflect about. The wire ends terminate at perimetral points 812 along the outer diameter of the frame but extend into the central axis of the frame 813. Pulling frame ends at points 813 in an axial direction away from the frame cause the beams to deflect down in an angle closer to parallel with the central axis of the frame. FIG. 104 is a fragmentary, perspective view of an occluder wire beam frame 814 that is similar to 811 and has a wire end restraint 815 component such as a crimp band with a central backing core.

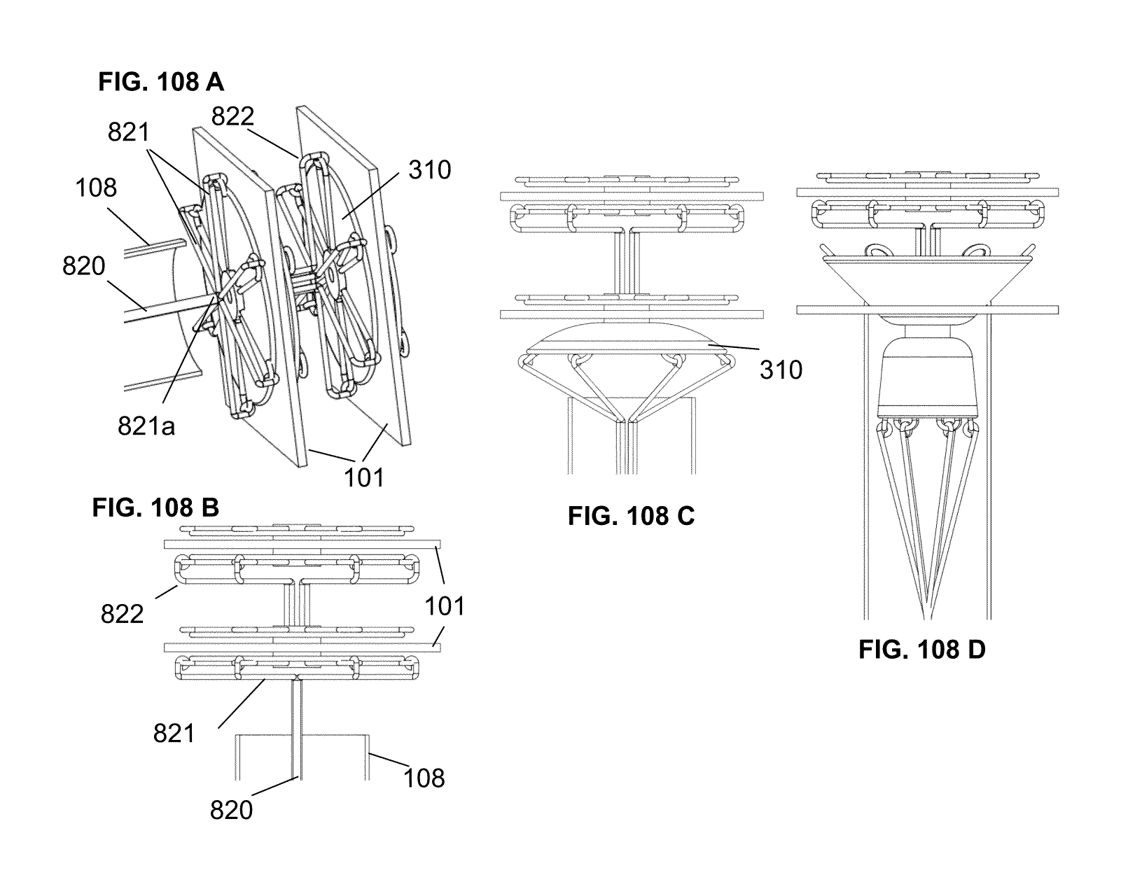

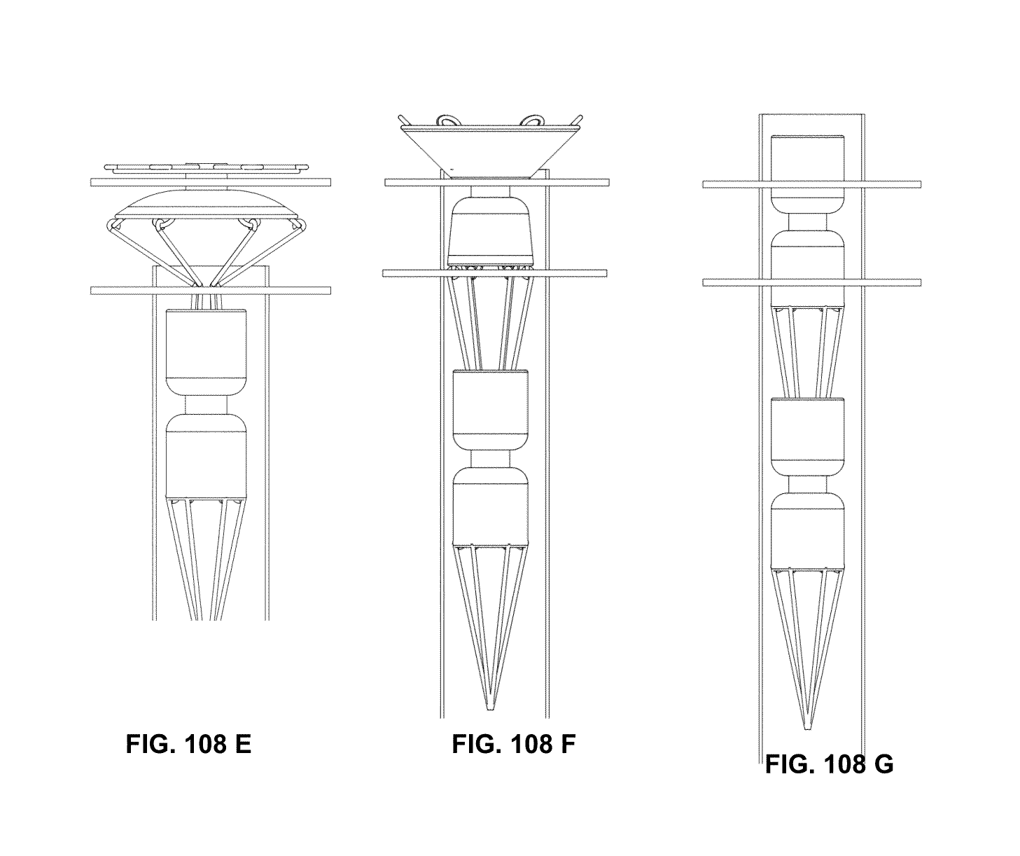

FIGS. 105, 106 and 107 are fragmentary, perspective views of occluder wire beam frames 816, 817 and 818 that are similar to occluder 814 but have different arrangement of continuous wire forms that use group transition sections 819 located offset from a central plane instead of an end restraint 815.

FIG. 108A is a fragmentary, perspective view of occluder sets with similar frames to occluder 800 shown in FIG. 94. Occluder frames have proximal group collapsing arms 821 that are attached to the outer periphery of the proximal occluder frame and extended toward the central axis and are shown to be grouped at a central point 821a attached to a flexible delivery member 820 for displacement relative to and into a delivery tube 108 that is similar to point 813 from FIG. 103. Occluders are shown expanded with sealing members 310 contacting vessel aperture walls 101. FIGS. 108B, 108C, 108D, 108E, 108F and 108G are fragmentary, side cross-sectional, diagrammatic views of a sequential recapturing or loading of an occluder set as shown in FIG. 108A, from apertures located within partial vessel walls 101 into an outertube 108. Actuation of occluder assembly into a collapsed state within a delivery tube 108 is done by grounding the delivery tube and pulling assembly from point 821a into delivery tube. Collapsing arms 822 are shown attached to the outer periphery of the most distal occluder similar to arms 821 and also attached to the central hub of the proximal occluder at their other end.

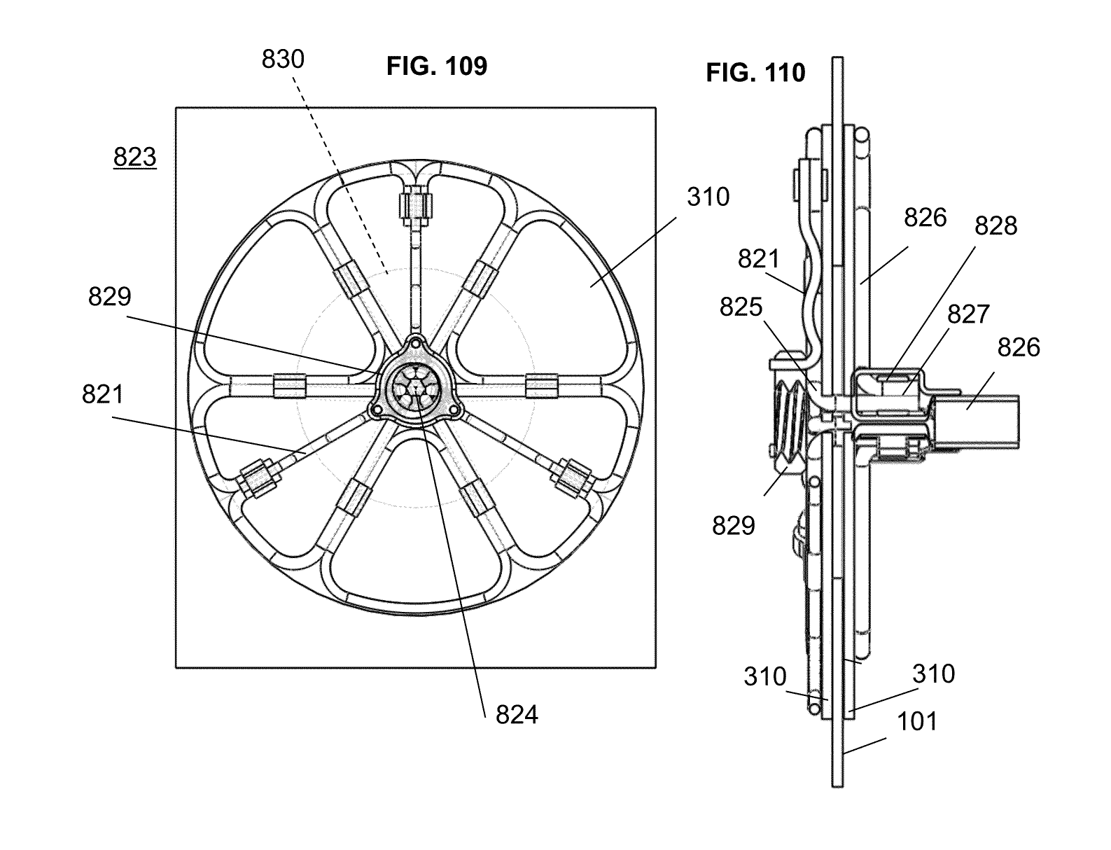

FIG. 109 is a proximal frontal view a wire frame occluder 823, including a sealing material 310, deployed into and about a vessel aperture 830 to complete occlude it, a closed central guidewire lumen 824, a connection member 829 attached to collapsing arms 821. FIG. 110 is a is a cross-sectional side view of occluder 823. Proximal beams 825 oppose distal beams 826 and their free wire ends culminate at point 827 and are constrained within a crimp band 828. In this view, the connection member 829 resides within the vessel and has a lower profile than the opposing side that contains frame attachment members and tether 826.

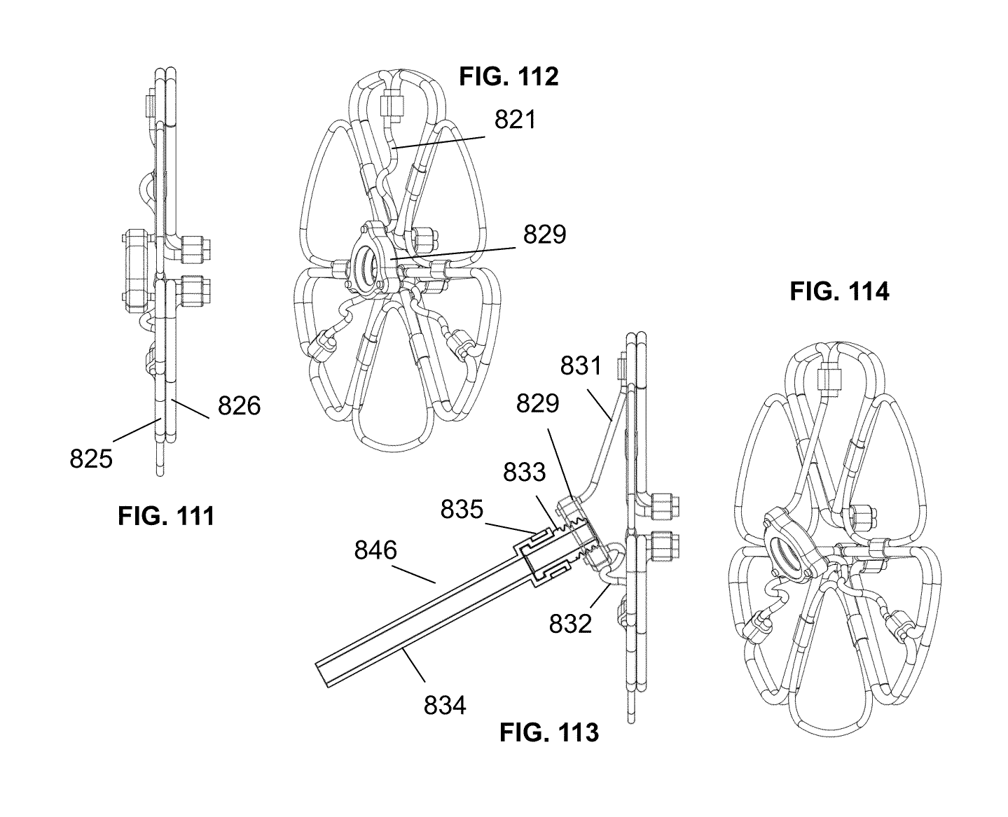

FIG. 111 is a fragmented side view of occluder 823 that demonstrates opposing beam groups 825 and 826 in their nominal positions contacting each other and containing no waist or gap length to achieve greater clamping preload. Alternatively, groups 825 and 826 can reside in the same plane or have a negative plane offset to achieve even greater preload.

FIG. 112 is a fragmented, perspective view of occluder 823 with connection member 829 and collapsing arms 821 in a nominal position. It can be appreciated from this view that collapsing arms 821 are configured as a spring-like serpentine shape having a total arc length greater than the distance between arm ends. Collapsing arms 821 are shown as separate components and attached to frame and connection member 829 but alternatively they can be extensions of the frame wires or extensions of the connection member.

FIG. 113 is a fragmented side view of occluder 823 demonstrating an articulated connection member 829 that is attached to a delivery member 836 using screw threads. Collapsing arms are able to deform under tension 831 and compression 832 to allow an angular difference between the delivery members center axis and the center axis of occluder frame. In this embodiment the delivery member 836 is shown in cross-section as an assembly composed of a main tube of a flexible material, a screw thread end 833 of a rigid material and a crimp band securing member 835, all having a central lumen to allow for guidewire insertion. FIG. 114 is a fragmented and perspective view of the occluder described in FIG. 113.

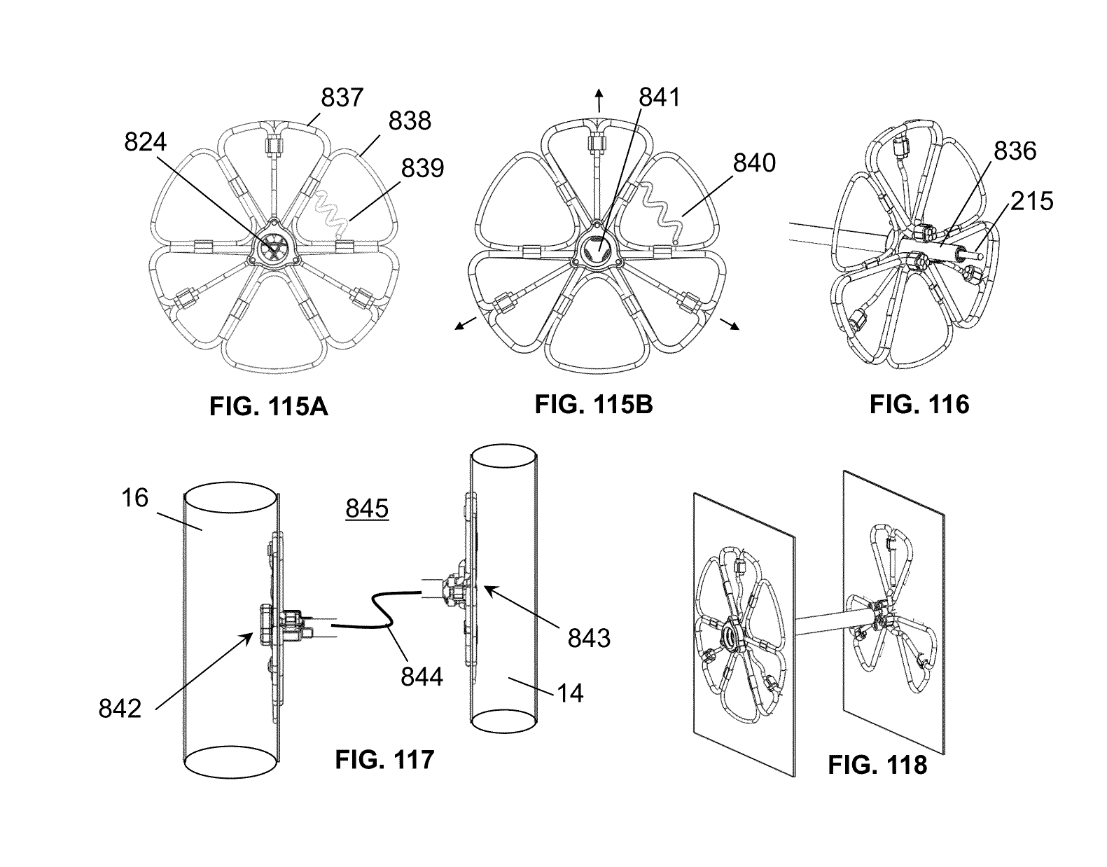

FIG. 115A is a fragmented proximal frontal view of a wire frame occluder 823 including a closed central guidewire lumen 824, an array of three frame main sections 837 that extend towards the central axis and an array of three frame spring sections 838. Sections 837 are spring loaded and forced into a radial direction by sections 838 shown diagrammatically by a tension spring 839 in a nominal position. In this embodiment, crimp-bands are used to constrain the ends of wire groups and are arranged similar to a collet, the crimp-bands are encased in a compliant sealing material to improve hemostasis while open and closed. Sealing material can be an extension of sealing disks, tether material or as independent components. In this embodiment closing force of guidewire lumen is provided in a radial direction that substantially opposes and is more perpendicular to fluid pressure that is presented in a axial direction to the guidewire lumen. FIG. 115B includes an open central guide wire lumen 841 shown diagrammatically by translating frame main sections 837 in the direction of the arrows. It can be observed that such translation is opposed by expanded tension spring 840 that pulls frame main sections towards each other thereby closing central guidewire lumen.

FIG. 116 is a fragmented perspective view of occluder 823 with a central guidewire lumen in an opened position by the insertion of guidewire support tube 836. Similarly, central guidewire lumen can be opened by the insertion of a guidewire 215 or other catheter type devices.

FIG. 117 is a fragmented perspective side view of an occluder pair 845 deployed into a vena cava type vessel 16 and abdominal aorta type vessel 14. Occluders are connected to each other by a diagrammatically represented tether 844. Points 842 and 843 demonstrate portions of the occluders located internal to vessel sides and the reduction of occluder volume compared to the outer vascular side that contains most of the occluder structural members.

FIG. 118 is a fragmented perspective view of an occluder pair 845 implanted onto vessel walls.

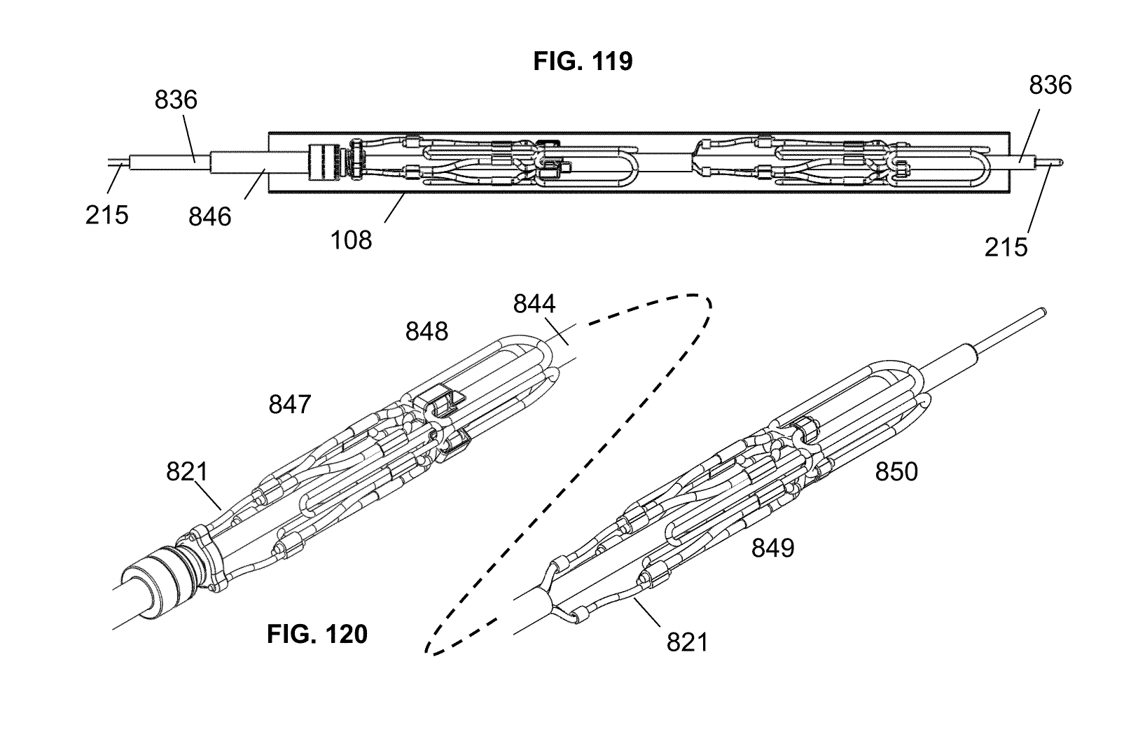

FIG. 119 is a fragmented side view of an occluder pair 845 in a collapsed configuration that contains a guidewire support tube 836, a guidewire 215, is attached to delivery member 846 and housed within an outer tube 108. Sealing materials can be housed within the gaps between the assembly and delivery tube.

FIG. 120 is a fragmentary and perspective view of occluder pair 845 shown in a collapsed state. Collapsing arms 821 are shown fully collapsed, proximal occluder groups 847 and 848 are shown fully collapsed and deflected away from each other to present a clamping zone and distal occluder groups 849 and 850 are shown fully collapsed and deflected away from each other to present a clamping zone.

Another exemplary embodiment of a wire form frame can be defined by a combination of wires or components to yield stiffer and less stiff sections to control, retention force, seal force, articulation, manipulation, ability to conform to anatomy, etc. This can be achieved by using different diameters along wire, different shaped profile wires, various materials, coils, braided wire, cables, and other rigid materials created by different manufacturing techniques such as machining or forming. Frame can also contain shaped sections, different profile sections or have additional components to improve or create sealing material attachment points and frame to frame sections attachment points. Attachment between occluder components can be achieved by using suture loops, pins, rivets, sandwich plats, clips, adhesive, a composite interweaved joint, and preset frame channels or loops attached to sealing material. Shaped sections can be in the form of loops or bends to capture sealing material attachment sutures. Attachment methods between frame and sealing material can be positioned such that they completely constrain frame and seal or allow for translation or freedom of movement between them. Similar configurations can be used in combination with all occluder components.

In other exemplary embodiments, frames can also contain shaped sections, different profile sections or have additional components such as bands, clips, barbs, anchors, and spikes to improve the anchoring or grip of the occluder to the vessel or tissue wall. Anchoring components can be attached to the frame, sealing material or other parts of the occluder independently. Anchor components can be configured such that traumatic sides are shielded up until occluder expansion to protect other neighboring components such at delivery tube or sealing materials.

Another exemplary embodiment of an occluder is that its structure is composed of a bladder having a collapsed deflated state, a partially inflated state, a fully inflated state, and an implanted state. The collapsed deflated state of the structure's size is adequate enough to pass through the vessel aperture. The partially inflated state allows for placement of the occluder. A fully inflated state allows full opposition of sealing surfaces by achieving preset interference geometries. The implanted state of the occluder is defined by a fully inflated bladder with preset interference or a partially inflated state where an operator determines adequate inflation. Additionally, the amount of inflation can be governed by volume or pressure. The bladder frame structure is globally sealed with one fill port opening to facilitate infusion of fluids. The bladder frame also can have another opening as an output port to serve as a transfer port for infusion fluids during fluid exchanges or to meter fill level. Temporary inflation can be done by a constantly liquid biocompatible material such as saline. Constant implantable inflation by the liquid material can be gained by selectably closing the fill port and the transfer port. Additionally, constant implantable inflation by way of fluid allows for deflation, occluder removal, and reentry at a later time. Fluid can be pulled into the reentry device or be absorbed into the body. Alternatively, an infusion medium that becomes solid, such as a two-part epoxy can be used to inflate the frame and will thereafter remain rigid without the use of valves. If the bladder frame is inflated by a fluid, it can be deflated by pulling a vacuum on the ports to remove the inflation fluid. A sealing material can be attached to the bladder frame or they can be one in the same by virtue of both members needing to be impermeable and flexible materials. Fluid transferred up to the occluder travels through channels that can also serve as attachment and detachment members to the delivery system by way of a user-controlled connection. Such connections can be press fit joints, screwed attachments, or have secondary release members. A hand-driven syringe or pump with reservoir feeds inflation channels.

Another exemplary embodiment of an occluder frame is a structure that is mechanically joined and able to translate from a collapsed state to a deployed state with preset interference geometries by way of a spring.

Another exemplary embodiment of an occluder frame is a structure that is mechanically joined and able to translate from a collapsed state to a deployed state by a driven self-locking mechanism, such as a screw and nut configuration. The mechanism is driven by a motion member in the delivery system and can be actuated to a preset interference geometry or to a user-defined geometry. The screw mechanism also can be actuated to translate the structure from a deployed state to a collapsed state. Alternatively, the occluder frame can be actuated by a composite of translations. For example, two rings, joined by pivoting linkages, have a nominal waist length set by linkage lengths. But, when the rings are twisted with respect to one another, the linkages begin to angle down and reduce the structure waist length down to zero.

Another exemplary embodiment of an occluder frame is a uniform structure that is nominal in its collapsed state and plastically deformed to a predetermined or user-defined interference geometry.

Another exemplary embodiment of an occluder frame is a structure that is a mechanically joined structure that can be collapsed in its nominal state then driven to a permanent predetermined or user-defined interference geometry by way of ratchet one-way locking mechanism.

Another exemplary embodiment of an occluder is a structure that translates from the collapsed state to the deployed state by any of the previously mentioned modalities and that is made from an impermeable material that facilitates sealing. This embodiment is a one-piece structure and seal.

Occluders can be made from the same machined tube, sheet, braided wire, extrusion and then fabricated to create a non-tensioning section.

Another exemplary occluder embodiment is a structure that translates from the collapsed state to the deployed state by any of the previously mentioned modalities that has a user adjustable preset geometry. Alternative to actively adjusting the occluder during implantation, an operator can preset geometries, such as interference gaps and diameter, on the bench before loading the occluder into the delivery device.

In greater detail, shape memory metallic frames can be made from flat sheet, tubes, braided, woven, and interweaved lattices then shape-set to preset geometries that are activated at or below body temperature. The shape memory material can be Nitinol. Lattice structure can also be fabricated by a combination of machining, laser cutting, joining, and welding of shape memory tubes or sheets.

In greater detail, plastically deformed metallic frames can be made from braided, woven, and interweaved lattices and then formed to final geometries when implanted. Metallic alloys can be stainless steel or cobalt chrome. The lattice structure also can be fabricated by a combination of machining, laser cutting, joining, and welding of metallic tubes or sheets.

In greater detail, the sealing material can be biological, such as harvested pericardium, to increase the biological similarities between the implant and the body, thus promoting ingrowth. In this case, the implant will be stored in solution to maintain profusion and natural material composition.

The sealing member also can be a laminated assembly with varying materials to promote both immediate and long-term seal integrity. A lamination of varying materials can also be configured to promote gradual endothelial growth.

In greater detail, a guidewire lumen can be formed by piercing of the occluder sealing material with the guidewire by the operator when loading the device. This action creates a pass through opening that is as small as possible. Structure frame members are sparse enough to not interfere with guidewire path and allow for an un-obstructed insertion. The guidewire lumen can be a patent opening in the occluder as designated by a structure frame or sealing material that allows for unobstructed preset pass-through of the guidewire.

The occluder set can be precisely deployed and translated from the collapsed configuration to the expanded configuration by using detents defining deployment stages. The user has to overcome the detents or lockout to initiate the sequential stages. A deployment mechanism can be used at the distal end of the device to precisely control deployment. Use of a threaded pusher allows for very fine control and mechanical advantage. A pushing mechanism at the distal end can be one-to-one and independent of friction and slop created by delivery system track.

Another exemplary embodiment of an occluder set is a set of occluders joined by a tether where the occluders and tether are specifically selected by an operator for patient geometries and assembled on the bench before loading onto the delivery system. Alternatively, a first occluder can be connected to a user-selectable release connection similar to the second occluder. This connection member can pass through or around the second occluder while in the collapsed, semi-expanded, and fully expanded states. This configuration does not rely on a permanent connection between the occluders. Additionally, the occluders can be loaded and delivered through separate systems.

Re-intervention through caval-aortic access can be achieved by including a re-entry or removal method as previously discussed with respect to FIGS. 32 to 34 and 71 to 74. A device similar to a deployment cable can be used to reconnect the occluder to the operator. Features such as magnets, hooks, and snares can be used for reattachment. Once a previously implanted occluder is secured, the user can re-collapse and retrieve the occluder to sequentially reintroduce the access conduit. Reentry through the implanted occluder can be achieved with the inclusion of a central occluder reentry plug as previously described with respect to FIGS. 32 to 34 and 71-74. A central aperture area is covered by an impermeable member that also conforms to sealing areas to create hemostasis. Alternatively, this central member can be impermeably attached to a dedicated sealing member that conforms to sealing surfaces. For the benefit of reintroduction, this sealing member can be configured to maintain hemostasis during the implanted condition but also allow for reintroduction by the application of opening forces applied by a reentry device. A user-applied and removed lock, such as suture, can be used to unlock and lock a gate. A central member can be a spring-loaded flap or a radially compressible material that allows a tapered introduction device to dilate. Alternatively, for the benefit of reintroduction, this central member can be selectably removed and an occluder structure frame can be limited to the perimeter to allow for an unobstructed reintroduction through the aperture. Once a secondary introduction is performed, a central sealing member can be reattached to both occluders. Connection between a selectably attached and removed central impermeable member to the occluder structure can be a threaded lock, attachment barbs, a radial force from the central member to occluder frame, a suture fixation, or a magnet. Alternatively, the central sealing member of the first and secondary occluder can be one in the same. Additionally, the occluder can intentionally dilate the aperture to allow the introducer sheath to fit within the central pass-through lumen of the occluder.

To increase endothelial growth, a growth solution can be irrigated by a user-operated syringe and through a lumen to eventually internally or externally irrigate the sealing material. In the embodiment where a fill bladder is used, intentional perforations in the bladder can allow a clotting/saline solution to escape during occluder deployment to accelerate endothelial growth.

Predetermined access to the internal surfaces of vessels and generally unoccupied interstitial space between vessels is advantageous to monitors that require access to blood flow such as pressure sensors, flow sensors, chemical sensors as demonstrated in FIGS. 69 and 70. Additionally, devices such as drug delivery valves can also reside within the vessel gap and have access to blood flow through the occluder.

Catheter assemblies need to be flushed with fluid to remove air within any existing lumens. A collapsed occluder with a perfect fit against the delivery tube and made from very impermeable material can prevent flushing of a central lumen. An internal delivery tube lumen with an irregular profile can intentionally cause fluid paths. An extruded section with irregular profile can be attached to a generally circular tube to form fluid path section. Alternatively, the delivery tube can contain array of holes to allow for fluid flow.

As a result of independent aperture sealing abilities, an occluder can be used in a device intended to seal one aperture in the body, such as a vessel, a natural orifice, a body entrance port, an organ entrance port, a repertory tract entrance port, a gastric tract entrance port, and/or a skin entrance port. Additionally, one occluder can be used to tie more than one tissue apertures together by constraining them within the occluder fixation mechanism. Additionally, occluders can have anchoring measures, such as threads, to attach other components to affix to the tissue occluder.

In an additional embodiment, vacuum can be used in the space between two vessels to bring vessels together and allow for a more controlled puncture and access into the second vessel. Alternatively relieving the vacuum or pressurizing will increase the space between vessels allowing more room for an occluder implantation. Vacuum and pressure can be transmitted through channels within delivery system or transmitted through a separate device.

A purpose designed transcaval guidewire can reduce procedural complications and increase operator precision and safety. The guidewire can have specific diameter sections to comply with stiffness and flexibility requirements of transcaval access. The guidewire can also have electrocautery compatible features such as an un-electrically insulated proximal end. An individual component can be made to connect the electro-cautery generator to the guidewire in a safe and effective manner. Such a device can be in the form of a clamp with correct guidewire contact features and a standard cautery plug or cable.

Additionally, transcaval access can be improved by using a purpose built guide catheter support structure as demonstrated in FIGS. 92 and 93. Current processes use off-label guide catheters to align guidewire with crossing point and yield unpredictable results. A device can be made to articulate and anchor the guide catheter during guidewire puncture and crossing. A catheter 700 with structural members 701 facilitates accurate alignment and support during guidewire 213 puncture. During insertion and manipulation, the catheter exhibits a generally circular cross-sectional profile along its longitudinal axis and is able to flex and conform during generally parallel translation throughout the central axis of vasculature. Operator can activate handle to deploy structural members 701 to articulate the distal end of the catheter throughout an angular range that can be perpendicular to vasculature central axis. Continued deployment of catheter structural members 701 can ground catheter to vessel 703 at points 702 and fix the guidewire 213 exit lumen to facilitate accurate crossing alignment that is not affected by fluid flow or straightening affect caused by guidewires that are stiffer than catheters. Alternatively, articulation and grounding can be achieved with separately controlled mechanisms or be provided in a separate device and used in conjunction with an existing guiding catheter.

The occluder deployment and implantation sequence has been described as first inserted into a venous tract and then an aortic tract; however, an alternate deployment sequence can be achieved by first inserting into the aortic tract and then the venous tract. Similarly, anatomical vessels, insertion locations and implantation locations can be used interchangeably wherever logically applicable.

Terms such as transcaval, TCA, TC, trans-caval, caval-aortic, aortocaval, aorto-caval, venous-arterial as used herein are the same. Terms such as aperture, opening, rent when used herein are the same. Terms such as tract, shunt, path when used herein are the same. Terms such as vessel, vessels, wall, walls, tissue, tissue wall, tissue walls, aortic vessel wall, venous vessel wall when used herein are the same.

Various descriptions of the occluder devices and of the closure methods have been used. Each of these descriptions is to be used interchangeably wherever logically applicable and is not to be limited to only one exemplary embodiment described or depicted.

It is noted that various individual features of the inventive processes and systems may be described only in one exemplary embodiment herein. The particular choice for description herein with regard to a single exemplary embodiment is not to be taken as a limitation that the particular feature is only applicable to the embodiment in which it is described. All features described herein are equally applicable to, additive, or interchangeable with any or all of the other exemplary embodiments described herein and in any combination or grouping or arrangement. In particular, use of a single reference numeral herein to illustrate, define, or describe a particular feature does not mean that the feature cannot be associated or equated to another feature in another drawing figure or description. Further, where two or more reference numerals are used in the figures or in the drawings, this should not be construed as being limited to only those embodiments or features, they are equally applicable to similar features or not a reference numeral is used or another reference numeral is omitted.

The foregoing description and accompanying drawings illustrate the principles, exemplary embodiments, and modes of operation of the systems and methods. However, the systems and methods should not be construed as being limited to the particular embodiments discussed above. Additional variations of the embodiments discussed above will be appreciated by those skilled in the art and the above-described embodiments should be regarded as illustrative rather than restrictive. Accordingly, it should be appreciated that variations to those embodiments can be made by those skilled in the art without departing from the scope of the systems and methods as defined by the following claims.

What is claimed is:

1. A vessel occluding assembly for occluding apertures in two vessels of a human, comprising:
a) a plurality of vessel aperture occluders each having an outer contact surface for interacting with a respective vessel aperture, wherein when each occluder is installed in its respective vessel aperture, hemostasis of the respective vessel is achieved,
the occluders having a collapsed state with a relatively reduced diameter sized for delivery at least partially through its respective aperture, and an expanded state with a relatively enlarged diameter for implantation and retention within the respective aperture; and
b) a flexible tether coupling the occluders together such that, when the two occluders are implanted in respective vessel apertures, the occluders achieve hemostasis of the vessels independent of the forces applied to tether between the occluders.

2. A vessel occluding assembly according to claim 1, wherein:
in the expanded state the occluder has opposite ends with a larger diameter, and a smaller diameter waist between the opposite ends.

3. A vessel occluding assembly according to claim 1, wherein:
each occluder includes an elastically deformable frame.

4. A vessel occluding assembly according to claim 3, wherein:
the deformable frame comprises nitinol.

5. A vessel occluding assembly according to claim 3, wherein:
the frame is formed from multiple wires.

6. A vessel occluding assembly according to claim 3, wherein:
the frame includes two groups of radial arrays of beams adapted for sandwiching a tissue wall between the two groups.

7. A vessel occluding assembly according to claim 6, wherein:
the two groups of radial array of beams are in a rotationally alternating configuration.

8. A vessel occluding assembly according to claim 6, wherein:
each of the two groups is provided with a sealing disk.

9. A vessel occluding assembly according to claim 3, wherein:
the frame is provided about a central hub.

10. A vessel occluding assembly according to claim 9, wherein:
the occluder has a central axis,
the frame is formed of at least one wire, and
the central hub is formed at least partly by portions of the at least one wire that together extend parallel to the central axis of the occluder, and a band surrounding the portions of the at least one wire.

11. A vessel occluding assembly according to claim 10, wherein:
the hub is providing with a sealing material.

12. A vessel occluding assembly according to claim 2, wherein:
the occluder includes a central axis, and a compliant and conforming sealing material is provided circumferentially about the central axis.

13. A vessel occluding assembly according to claim 12, wherein:
the sealing material is in the form of a skirt with spaced protuberances about its periphery.

14. A vessel occluding assembly according to claim 1, further comprising:
at least one of a guidewire, a guidewire support tube, and a catheter,
wherein the occluders each include a respective opening, and the at least one of the guidewire, guidewire support tube, and/or catheter is received through the openings.

15. A vessel occluding assembly according to claim 14, wherein:
the occluders include a closure for automatically closing the openings upon removal of the guidewire, guidewire support tube, and/or catheter.

16. A vessel occluding assembly according to claim 14, wherein:
the occluders define a central longitudinal axis, and the guidewire passage is located off-axis from said central longitudinal axis.

17. A vessel occluding assembly according to claim 1, wherein:
the occluders each include a frame, and each frame includes a central hub and a plurality of beams arranged about the central hub, wherein for each occluder,
in the collapsed state, the beams are provided in first and second groups of beams, and the first group is directed substantially opposite and away from the second group relative to the hub, and
in the expanded state the first and second groups of beams radially extend in relation to the hub.

18. A vessel occluding assembly according to claim 17, further comprising:
in the collapsed state, the second set of beams of a first occluder of the occluder set are arranged in an interleaved configuration with the first set of beams of the first occluder of the occluder set.

19. A vessel occluding assembly according to claim 17, wherein:
each of the first and second group of beams is provided with a sealing member integrated with the first and second group of beams.

20. A vessel occluding assembly according to claim 19, wherein:
in the collapsed state, the sealing member forms a pleated configuration.

21. A vessel occluding assembly according to claim 17, wherein:
in the expanded state the beams are flat.

22. A vessel occluding assembly according to claim 17, wherein:
in the expanded state the beams are bent.

23. A vessel occluding assembly according to claim 1, wherein:
one of the occluders includes a sensor coupled thereto, and a blood path to convey blood within the vessel at which the occluder is coupled to the sensor.

24. A vessel occluding assembly according to claim 1, wherein:
at least one of the occluders has a non-circular shape.

25. A vessel occluding assembly according to claim 1, wherein:
at least one of the occluders has a curved geometric portion to conform to curved geometries of an inner and outer tubular vessel wall.

26. A vessel occluding assembly according to claim 1, wherein:
the reduced diameter of the collapsed state is sized for delivery through the femoral vein.

27. A system for occluding apertures in the walls of two vessels, comprising:
a) the vessel occluding assembly of claim 1; and
b) a delivery system for delivering the vessel occluding assembly into the apertures of the two vessels, the delivery system including,
i) an elongate flexible delivery tube having a proximal end, a distal end, and an outer diameter, the distal end sized to receive the vessel occluding assembly and retain the occluders in the collapsed state,
ii) an elongate flexible delivery member having a proximal end and distal end, the delivery member extending through and longitudinally displaceable relative to the delivery tube, and
iii) a connection member provided at the distal end of the delivery member that is coupled for temporary attachment and release to the vessel occluding assembly.

28. A system according to claim 27, wherein the delivery tube has a curve at its distal end.

29. A system according to claim 27, wherein the delivery tube has an obliquely angled cut at its distal end.

30. A deployment system for a vessel occluding assembly for apertures in vessel walls, comprising:
a) an elongate flexible delivery tube having a proximal end, a distal end, and an outer diameter, the distal end sized to receive the vessel occluding assembly and retain the occluders in the collapsed state, the outer diameter sized to be received within the femoral vein,
b) an elongate flexible delivery member having a proximal end and distal end, the delivery member extending through and longitudinally displaceable relative to the delivery tube, the delivery member having a connection member provided at the distal end of the delivery member that is coupled for temporary attachment and release to the vessel occluding assembly; and
c) an anti-pullout mechanism for selectively restricting movement of the delivery member relative to the delivery tube.

31. An occluder for sealing an aperture in a tissue wall, the tissue wall having opposite sides, comprising:
a) a deformable wire frame member insertable into the aperture; and
b) a seal member adapted to contact opposite sides of the tissue wall to form a seal,
the occluder having a collapsed state with a relatively reduced diameter sized for delivery at least partially through the aperture, and an expanded state with a relatively enlarged diameter for implantation and retention about the aperture.

32. An occluder according to claim 31, wherein:
the frame is formed from a multiple wires.

33. An occluder according to claim 31, wherein:
the frame includes a central hub and a plurality of structural beams arranged about the central hub, and
in the collapsed state, the beams are provided in first and second groups of beams, and the first group is directed substantially opposite and away from the second group relative to the hub, and
in the expanded state the first and second groups of beams radially extend in relation to the hub.

34. An occluder according to claim 33, further comprising:
in the collapsed state, the second set of beams is arranged in an interleaved configuration with the first set of beams.

35. An occluder according to claim 33, wherein:
the two groups of beams are in a rotationally alternating configuration.

36. An occluder according to claim 31, wherein:
the seal member is a sealing disk provided to each of the two groups of beams.

37. An occluder according to claim 31, wherein:
the frame is provided about a central hub.

38. An occluder according to claim 37, wherein:
the occluder has a central axis,
the frame is formed of at least one wire, and
the central hub is formed at least partly by portions of the at least one wire that together extend parallel to the central axis of the occluder, and a band surrounding the portions of the at least one wire.

39. An occluder according to claim 37, further comprising:
at least one of a guidewire, a guidewire support tube, and a catheter,
wherein the occluder includes a respective opening, and the at least one of the guidewire, guidewire support tube, and/or catheter is received through the openings.

40. An occluder according to claim 39, wherein:
the occluder includes a closure for automatically closing the opening upon removal of the guidewire, guidewire support tube, and/or catheter.

41. A system for occluding an aperture in a tissue wall, comprising:
a) the vessel occluder of claim 31; and
b) a delivery system for delivering the vessel occluder in the aperture in the tissue wall, the delivery system including,
i) an elongate flexible delivery tube having a proximal end, a distal end, and an outer diameter, the distal end sized to receive the vessel occluder and retain the occluder in the collapsed state,
ii) an elongate flexible delivery member having a proximal end and distal end, the delivery member extending through and longitudinally displaceable relative to the delivery tube, and
iii) a connection member provided at the distal end of the delivery member that is coupled for temporary attachment and release to the vessel occluder.

42. An electrocautery guidewire system, comprising:
a) a guidewire having a proximal portion with a conductive section; and
b) an electrocautery guidewire adapter, including:
i) a clamp having an atraumatic engagement portion that couples to the guidewire and a conductive portion that contacts the conductive section of the guidewire,
ii) a hand-operated actuation portion to release the engagement portion from the guidewire, and
iii) a cautery electrical connection that couples a cautery source to the conductive portion of the clamp, and consequently to the guidewire.

43. A method of occluding apertures in the walls of first and second vessels, the first vessel having a first aperture in its vessel wall, and the second vessel having a second aperture in its vessel wall, comprising:
a) providing a vessel occluding assembly includes a first and second vessel aperture occluders, each having a vessel aperture outer contact surface, and a flexible tether connecting the first and second occluders together;
b) implanting the first occluder in the first aperture to achieve hemostasis; and
c) implanting the second occluder in the second aperture to achieve hemostasis, wherein when the first and second occluders are implanted in their respective apertures, the occluders achieve hemostasis of the first and second vessels independent of a relative tension between the first and second vessels.

44. A method according to claim 43, wherein:
when the first and second occluders achieve hemostasis, the tether is slack.

45. A method according to claim 44, wherein:
prior to implanting the first and second occluders, a pre-implantation interstitial gap width is provided between the first and second vessels, and
after implanting the first and second occluders, a post-implantation interstitial gap width is provided between the first and second vessels,
the pre-implantation interstitial gap width and the post-implantation interstitial gap width are substantially the same.

46. A method according to claim 43, wherein:
the first occluder is implanted before the second occluder is implanted.

47. A method according to claim 43, wherein:
implanting the first occluder includes,
i) insertion of the first occluder through the second aperture and within the first aperture,
ii) partial expansion of the first occluder within the first aperture,
iii) full expansion of the first occluder; and
implanting the second occluder includes,
i) partial expansion of the second occluder, and
ii) full expansion of the second occluder.

48. A method according to claim 43, wherein:
the first occluder includes a frame having a first group of beams and a second group of beams, and in a collapsed delivery configuration, the first group is preloaded to be directed substantially opposite the second group, with a vessel wall capture zone defined therebetween, and wherein,
implanting the first occluder includes,
i) inserting the first occluder within the first aperture until the vessel wall of the first vessel is within the vessel wall capture zone,
ii) partial expansion of the first occluder within the first aperture such that the first group of beams assumes an expanded shape on a first side of the vessel wall of the first vessel, and
iii) full expansion of the first occluder such that the second group of beams assumes an expanded shape on a second side of the vessel wall of the first vessel and the vessel wall is captured in the vessel wall capture zone between the first and second groups of beams.
